SEQUENCE LISTING (50 sequences)

SEQ ID NO: 1 - protein, 4 aa, synthetic construct
  REGION 1..4: note = targeting motif
RGRR

SEQ ID NO: 2 - protein, 5 aa, synthetic construct
  REGION 1..5: note = targeting motif
RGRRS

SEQ ID NO: 3 - protein, 5 aa, synthetic construct
  REGION 1..5: note = targeting motif
YQLDV

SEQ ID NO: 4 - protein, 4 aa, synthetic construct
  REGION 1..4: note = targeting motif
VGVA

SEQ ID NO: 5 - protein, 8 aa, synthetic construct
  REGION 1..8: note = hydrophilic amino acid sequence including a targeting agent
GHGKHKNK

SEQ ID NO: 6 - protein, 9 aa, synthetic construct
  REGION 1..9: note = hydrophilic amino acid sequence including a targeting agent
CRGDKGPDC

SEQ ID NO: 7 - protein, 7 aa, synthetic construct
  REGION 1..7: note = hydrophilic amino acid sequence including a targeting agent
CKGAKAR

SEQ ID NO: 8 - protein, 9 aa, synthetic construct
  REGION 1..9: note = hydrophilic amino acid sequence including a targeting agent
CRVSRQNKC

SEQ ID NO: 9 - protein, 9 aa, synthetic construct
  REGION 1..9: note = hydrophilic amino acid sequence including a targeting agent
CGGERGKSC

SEQ ID NO: 10 - protein, 6 aa, synthetic construct
  REGION 1..6: note = hydrophilic amino acid sequence including a targeting agent
CRSRKG

SEQ ID NO: 11 - protein, 6 aa, synthetic construct
  REGION 1..6: note = hydrophilic amino acid sequence including a targeting agent
CKAAKN

SEQ ID NO: 12 - protein, 7 aa, synthetic construct
  REGION 1..7: note = hydrophilic amino acid sequence including a targeting agent
CRGRRST

SEQ ID NO: 13 - protein, 8 aa, synthetic construct
  REGION 1..8: note = hydrophilic amino acid sequence including a targeting agent
CEYQLDVE

SEQ ID NO: 14 - protein, 7 aa, synthetic construct
  REGION 1..7: note = hydrophilic amino acid sequence including a targeting agent
TVRTSAD

SEQ ID NO: 15 - protein, 7 aa, synthetic construct
  REGION 1..7: note = hydrophilic amino acid sequence including a targeting agent
PIEDRPM

SEQ ID NO: 16 - protein, 7 aa, synthetic construct
  REGION 1..7: note = hydrophilic amino acid sequence including a targeting agent
ALRDRPM

SEQ ID NO: 17 - protein, 7 aa, synthetic construct
  REGION 1..7: note = hydrophilic amino acid sequence including a targeting agent
PEKFRPM

SEQ ID NO: 18 - protein, 7 aa, synthetic construct
  REGION 1..7: note = hydrophilic amino acid sequence including a targeting agent
IKVGKLQ

SEQ ID NO: 19 - protein, 12 aa, synthetic construct
  REGION 1..12: note = hydrophilic amino acid sequence including a targeting agent
SVSVGMKPSPRP

SEQ ID NO: 20 - protein, 7 aa, synthetic construct
  REGION 1..7: note = hydrophilic amino acid sequence including a targeting agent
VPEQRPM

SEQ ID NO: 21 - protein, 9 aa, synthetic construct
  REGION 1..9: note = hydrophilic amino acid sequence including a targeting agent
CAKIDPELC

SEQ ID NO: 22 - protein, 9 aa, synthetic construct
  REGION 1..9: note = hydrophilic amino acid sequence including a targeting agent
CSNIDARAC

SEQ ID NO: 23 - protein, 6 aa, synthetic construct
  REGION 1..6: note = hydrophilic amino acid sequence including a targeting agent
RLQLKL

SEQ ID NO: 24 - protein, 8 aa, synthetic construct
  REGION 1..8: note = hydrophilic amino acid sequence including a targeting agent
PMMRQRPM

SEQ ID NO: 25 - protein, 7 aa, synthetic construct
  REGION 1..7: note = hydrophilic amino acid sequence including a targeting agent
AKATCPA

SEQ ID NO: 26 - protein, 7 aa, synthetic construct
  REGION 1..7: note = hydrophilic amino acid sequence including a targeting agent
QPPMEYS

SEQ ID NO: 27 - protein, 7 aa, synthetic construct
  REGION 1..7: note = hydrophilic amino acid sequence including a targeting agent
SISSLTD

SEQ ID NO: 28 - protein, 8 aa, synthetic construct
  REGION 1..8: note = hydrophilic amino acid sequence including a targeting agent
FRVGVADV

SEQ ID NO: 29 - protein, 13 aa, synthetic construct
  REGION 1..13: note = hydrophilic amino acid sequence including a targeting agent
CNGRCVSGCAGRC

SEQ ID NO: 30 - protein, 7 aa, synthetic construct
  REGION 1..7: note = hydrophilic amino acid sequence including a targeting agent
NWGDRIL

SEQ ID NO: 31 - protein, 9 aa, synthetic construct
  REGION 1..9: note = hydrophilic amino acid sequence including a targeting agent
CVSNPRWKC

SEQ ID NO: 32 - protein, 9 aa, synthetic construct
  REGION 1..9: note = hydrophilic amino acid sequence including a targeting agent
CDCRGDCFC

SEQ ID NO: 33 - protein, 12 aa, synthetic construct
  REGION 1..12: note = hydrophilic amino acid sequence including a targeting agent
YSAYPDSVPMMS

SEQ ID NO: 34 - protein, 7 aa, synthetic construct
  REGION 1..7: note = hydrophilic amino acid sequence including a targeting agent
PLASRPM

SEQ ID NO: 35 - protein, 5 aa, synthetic construct
  REGION 1..5: note = hydrophilic amino acid sequence including a targeting agent
KGRGD

SEQ ID NO: 36 - protein, 4 aa, synthetic construct
  REGION 1..4: note = hydrophilic amino acid sequence including an RGD targeting agent
RGDS

SEQ ID NO: 37 - protein, 4 aa, synthetic construct
  REGION 1..4: note = hydrophilic amino acid sequence including an RGD targeting agent
GRGD

SEQ ID NO: 38 - protein, 5 aa, synthetic construct
  REGION 1..5: note = hydrophilic amino acid sequence including an RGD targeting agent
GRGDS

SEQ ID NO: 39 - protein, 6 aa, synthetic construct
  REGION 1..6: note = hydrophilic amino acid sequence including an RGD targeting agent
GRGDSP

SEQ ID NO: 40 - protein, 7 aa, synthetic construct
  REGION 1..7: note = hydrophilic amino acid sequence including an RGD targeting agent
GRGDSPK

SEQ ID NO: 41 - protein, 6 aa, synthetic construct
  REGION 1..6: note = hydrophilic amino acid sequence including an RGD targeting agent
GRGDNP

SEQ ID NO: 42 - protein, 6 aa, synthetic construct
  REGION 1..6: note = hydrophilic amino acid sequence including an RGD targeting agent
GRGDTP

SEQ ID NO: 43 - protein, 9 aa, synthetic construct
  REGION 1..9: note = targeting motif
CRKRLDRNC

SEQ ID NO: 44 - protein, 12 aa, synthetic construct
  REGION 1..12: note = targeting motif
EFEEFEIDEEEK

SEQ ID NO: 45 - protein, 11 aa, synthetic construct
  REGION 1..11: note = targeting motif
DFEEIPEEYLQ

SEQ ID NO: 46 - protein, 7 aa, synthetic construct
  REGION 1..7: note = cross-linking motif
GGGCCGG

SEQ ID NO: 47 - protein, 15 aa, synthetic construct
  REGION 1..15: note = amphiphilic peptide
  REGION 1..3: note = X - Each X is pentafluoro phenylalanine (2,3,4,5,6-pentafluoro-L-phenylalanine).
XXXGGGCCGGKGRGD

SEQ ID NO: 48 - protein, 5 aa, synthetic construct
  REGION 1..5: note = C-terminally amidated hydrophilic amino acid sequence which contains targeting motif RGD
  MOD_RES 5: note = AMIDATION -
KGRGD

SEQ ID NO: 49 - protein, 15 aa, synthetic construct
  REGION 1..15: note = amphiphilic peptide
  REGION 1..3: note = X - Each X is pentafluoro phenylalanine (2,3,4,5,6-pentafluoro-L-phenylalanine).
  SITE 15: note = AMIDATION
XXXGGGCCGGKGRGD

SEQ ID NO: 50 - protein, 6 aa, synthetic construct
  REGION 1..6: note = hydrophilic amino acid sequence including a targeting agent
CRGRRT

(19) United States
(12) Patent Application Publication
(10) Pub. No.: US 2026/0014283 A1
(43) Pub. Date: Jan. 15, 2026

(54) ULTRASOUND-SENSITIVE PEPTIDE PARTICLES FOR SPATIALLY RESOLVED MOLECULE DELIVERY AND METHODS OF USING THE SAME

(71) Applicant: The Penn State Research Foundation, State College, PA (US)

(72) Inventor: Scott H. Medina, State College, PA (US)

(21) Appl. No.: 19/201,385

(22) Filed: May 7, 2025

Related U.S. Application Data
(60) Provisional application No. 63/670,462, filed on Jul. 12, 2024.

(51) Int. Cl. A61K 49/22; A61K 47/64

(52) U.S. Cl. CPC A61K 49/225; A61K 47/645; A61K 49/221

(57) ABSTRACT

Provided herein are compositions comprising cells that each comprise at least one peptide-based nanoparticle. In some embodiments, the peptide-based nanoparticles each comprise a perfluorocarbon liquid core and a plurality of amphiphilic peptides surrounding the perfluorocarbon liquid core. Also provided herein are methods of preparing any of the compositions described herein, as well as methods of cellular tracking using any of the composition described herein.

### CROSS-REFERENCE TO RELATED APPLICATION

This application claims the benefit of priority of U.S. Provisional Patent Application Ser. No. 63/670,462, filed on Jul. 12, 2024, entitled “ULTRASOUND-SENSITIVE PEPTIDE PARTICLES FOR SPATIALLY RESOLVED MOLECULE DELIVERY AND METHODS OF USING THE SAME,” the entire contents of which is hereby incorporated by reference herein.

### GOVERNMENT LICENSE RIGHTS

This invention was made with government support under Grant Nos. DK128638 and GM142903 awarded by National Institutes of Health and under Grant No. DMR1845053 awarded by the National Science Foundation. The Government has certain rights in the invention.

### SEQUENCE LISTING

This application contains a computer readable Sequence Listing which has been submitted in XML file format with this application, the entire content of which is incorporated by reference herein in its entirety. The Sequence Listing XML file submitted with this application is entitled “148411004300 Sequence Listing.xml”, was created on Jul. 11, 2024, and is 66,120 bytes in size.

### FIELD OF THE INVENTION

The present invention is directed to fluorous phase-directed peptide assemblies capable of being triggered by ultrasound. It further pertains to methods for acoustically activating phase-changing nanoparticles within cells, permitting in situ imaging and targeting various cell types for specific applications.

### BACKGROUND OF THE INVENTION

Protein and nucleic-acid based agents are some of the most potent therapeutic tools used in precision medicine. They deliver biologically active cargo to target cells and are revolutionizing the treatment of various human diseases, including cancer, autoimmune disorders and diabetes. However, therapeutic utility of these biomacromolecules is limited by their ineffective distribution to diseased tissues, poor stability in physiologic environments, and toxic side effects. Importantly, many large biomolecules are unable to penetrate into cells and, therefore, existing biotherapeutics have been restricted to disease targets displayed on the cell surface. Due to the large size, traditional biocarriers rely on endocytic transport processes to enter diseased cells, ultimately leading to degradation of delivered biologics in the harsh endosomal environment. The cell-targeted biocarriers also require multi-step harsh physical and chemical formulation methods that ultimately render biological cargo inactive.

Dynamic peptide assemblies can be formed as a result of a spontaneous self-sorting and assembly process. The architecture and behavior of these self-assembled peptide structures can be controlled by modulating the physicochemical properties of the building blocks, environmental conditions, or assembly kinetics. This has resulted in a wide variety of ordered peptide arrangements including sheets, fibrils, and tubes, which are playing increasingly important roles in the formation of biomaterials and biomedical devices. While various peptide assemblies have been described in the literature, there have been no reports of using these peptide assemblies as drug delivery vehicles for delivering, for example, protein and nucleic acid-based therapeutic agents into cells.

On the contrary, currently known protein delivery vehicles transport proteins intracellularly using the endocytic pathway whereby the vehicle must undergo endosomal escape for successful cytoplasmic delivery. Endosomal escape requires proton pumping into the endosome which lowers the pH and can lead to protein denaturation. A delivery vehicle capable of direct intracellular delivery, bypassing the endocytic pathway, is favorable for effective protein therapy. In light of the valuable therapeutic potential of protein and nucleic acid based therapeutic compounds, effective compositions and methods to introduce such compounds into cells is needed in the art.

Recognizing similar challenges in cellular therapies has prompter a parallel need for advanced imaging techniques to monitor immune cells in vivo. Macrophages, key players in both innate and adaptive immunity, are critical for wound healing, tissue regeneration, and are implicated in a range of inflammatory diseases including diabetes, atherosclerosis, and rheumatoid arthritis. Their diverse functions and roles in disease modulation make them significant targets for imaging and therapeutic interventions. However, challenges persist in the effective monitoring and manipulation of macrophages due to their complex biodistribution and migration patterns, which can lead to heterogenous therapeutic outcomes and increased risk of side effects. Developing non-invasive, real-time imaging methods that offer detailed spatial resolution and prolonged monitoring capabilities is essential to better understand macrophage behavior and improve therapeutic outcomes.

### SUMMARY OF THE INVENTION

Disclosed herein are compositions comprising a plurality of cells, wherein each cell comprises at least one peptide-based nanoparticle. In some embodiments, the at least one peptide-based nanoparticle comprises a perfluorocarbon liquid core and a plurality of amphiphilic peptides surrounding the perfluorocarbon liquid core, wherein each amphiphilic peptide is represented by Formula (III):

HB-CL-HP—NH2 (III)

wherein HB is a fluorinated hydrophobic block consisting of three to five consecutively connected pentafluorinated hydrophobic amino acid residues; wherein CL is an amino acid sequence consisting of two to 10 amino acid residues, at least two of which are cross-linking cysteine residues; wherein HP is a hydrophilic amino acid sequence, wherein said amphiphilic peptide consists of 8 to 30 total amino acid residues, and wherein the amphiphilic peptides are oriented such that groups HB of the amphiphilic peptides are interpolated into the perfluorocarbon liquid of the perfluorocarbon liquid core and groups HP extend away from the perfluorocarbon liquid core.

In some embodiments, each cell of the plurality of cells is a macrophage.

In some embodiments, the amphiphilic peptides of each peptide-based nanoparticle are crosslinked to each other through the cross-linking motif CL. In some embodiments, each peptide-based nanoparticle comprises a crosslinked unimolecular monolayer morphology or a 2D sheet morphology. In some embodiments, the amphiphilic peptides of each peptide-based nanoparticle are not crosslinked to each other. In some embodiments, each peptide-based nanoparticle comprises a non-crosslinked unimolecular monolayer morphology or a ID fibrils morphology.

In some embodiments, the composition further comprises a pharmaceutically acceptable excipient selected from the group consisting of a vehicle, an adjuvant, a carrier, and a diluent.

In some embodiments, HB consists of three, four or five consecutively connected pentafluoro-phenylalanine residues, and is located at the N-terminal end of the peptide sequence. In some embodiments, HP comprises lysine, glycine, arginine, aspartic acid, or any combination thereof. In some embodiments, HP comprises the sequence KGRGD (SEQ ID NO: 35), where K is lysine, G is glycine, R is arginine, and D is aspartic acid. In some embodiments, CL comprises GGGCCGG (SEQ ID NO: 46), where G is glycine and C is cysteine. In some embodiments, the hydrophilic amino acid sequence of HP comprises a targeting motif. In some embodiments, the hydrophilic amino acid sequence comprises a conserved targeting motif selected from the group consisting of: HGK, RGD, KAR, RSR, KAA, RGRR(SEQ ID NO:1), RGRRS (SEQ ID NO:2), YQLDV (SEQ ID NO:3), EYQ, RPM, PSP, VGVA (SEQ ID NO:4), NGR, CRKRLDRNC (SEQ ID NO:43), EFEEFEIDEEEK (SEQ ID NO:44), and DFEEIPEEYLQ (SEQ ID NO:45). In some embodiments, the hydrophilic amino acid sequence comprises a hydrophilic amino acid sequence selected from the group consisting of: KGRGD (SEQ ID NO:35), RGDS (SEQ ID NO:36), GRGD (SEQ ID NO:37), GRGDS (SEQ ID NO:38), GRGDSP (SEQ ID NO:39), GRGDSPK (SEQ ID NO:40), GRGDNP (SEQ ID NO:41), and GRGDTP (SEQ ID NO:42).

In some embodiments, the amphiphilic peptide comprises an amphiphilic peptide represented by Formula (IV) or Formula (V): FFFFFFGGGCCGGKGRGD (IV) (SEQ ID NO:47), FFFFFFFFGGGCCGGKGRGD-NH2 (V) (SEQ ID NO:49), wherein FF is pentafluoro-phenylalanine, G is glycine, C is cysteine, K is lysine, G is glycine, R is arginine, and D is aspartic acid.

In some embodiments, the amino acid sequence of CL consists of two to 10 amino acid residues and said hydrophilic amino acid sequence of HP consists of 3 to 15 hydrophilic amino acids, and wherein said amphiphilic peptide consists of 10 to 30 total amino acid residues.

In some embodiments, the amphiphilic peptide has a molecular weight in a range of about 2000-5000 daltons, wherein the amphiphilic peptide comprises at least eight amino acid residues, and a total number of no more than 30 amino acid residues, wherein at least two of the amino acid residues are connected consecutively by peptide bonds without any intervening amino acid residues.

In some embodiments, the composition comprising a plurality of macrophages, wherein each macrophage comprises at least one peptide-based nanoparticle, wherein the at least one peptide-based nanoparticle comprises a perfluorocarbon liquid core and a plurality of amphiphilic peptides surrounding the perfluorocarbon liquid core, wherein each amphiphilic peptide comprises FFFFFFFFGGGCCGGKGRGD-NH2 (SEQ ID NO: 49), wherein FF is pentafluoro-phenylalanine, G is glycine, C is cysteine, K is lysine, G is glycine, R is arginine, and D is aspartic acid, and wherein amphiphilic peptides are oriented such that the FFFFFFFF region is interpolated into the perfluorocarbon liquid core and the KGRGD region extends away from the perfluorocarbon liquid.

In some embodiments, the amphiphilic peptides of each peptide-based nanoparticle are crosslinked to each other through a cross-linking motif GGGCCGG, and wherein each peptide-based nanoparticle comprises a crosslinked unimolecular monolayer morphology or a 2D sheet morphology. In some embodiments, the amphiphilic peptides of each peptide-based nanoparticle are not crosslinked to each other, and wherein each peptide-based nanoparticle comprises a non-crosslinked unimolecular monolayer morphology or a ID fibrils morphology.

Also provided herein are methods of preparing any cellular compositions comprising a plurality of cells described herein. In some embodiments, the method comprises contacting a perfluorocarbon liquid with a plurality of amphiphilic peptides to form a plurality of peptide-based nanoparticles, wherein each peptide-based nanoparticle comprises a perfluorocarbon liquid core and a plurality of amphiphilic peptides surrounding the perfluorocarbon liquid core, and contacting the plurality of peptide-based nanoparticles with the plurality of cells, wherein each cell of the plurality of cells internalizes at least one peptide-based nanoparticle.

In some embodiments, water is added to plurality of peptide-based nanoparticles after their formation. In some embodiments, the amphiphilic peptides of each peptide-based nanoparticle are crosslinked to each other, forming a peptide-based nanoparticle comprising a crosslinked unimolecular monolayer morphology. In some embodiments, the amphiphilic peptides of each peptide-based nanoparticle are not crosslinked to each other, forming a peptide-based nanoparticle comprising a non-crosslinked unimolecular monolayer morphology.

In some embodiments, water is added to the plurality of amphiphilic peptides prior to their contact with the perfluorocarbon liquid. In some embodiments, the amphiphilic peptides of each peptide-based nanoparticle are crosslinked to each other, forming a peptide-based nanoparticle comprising a 2D sheet morphology. In some embodiments, the amphiphilic peptides of each peptide-based nanoparticle are not crosslinked to each other, forming a peptide-based nanoparticle comprising a ID fibrils morphology.

Also provided herein are methods of cellular tracking. In some embodiments, the method comprises administrating any composition described herein to a tissue, administrating ultrasonic waves to the tissue, and detecting the location of the cells in the tissue by locating acoustic properties of the peptide-based nanoparticles in the plurality of cells. In some embodiments, the ultrasonic waves are administered to the tissue by a B-mode ultrasonic imaging device or a Doppler ultrasonic imaging device. In some embodiments, the ultrasonic waves induce a liquid-to-gas phase transition in the peptide-based nanoparticles that generates echogenic microbubbles inside the plurality of cells.

### BRIEF DESCRIPTION OF THE DRAWINGS

The above and other aspects and features of the present disclosure will become more apparent in the following detailed description when taken in conjunction with reference to the accompanying drawings, in which:

FIG. 1 is a diagram showing structure of nanoparticles according to embodiments.

FIG. 2 is a diagram showing target motif-mediated specific binding and ultrasound-mediated delivery of cargo into a targeted cell.

FIG. 3 is a graph of particle size (nanometers, nm) and count rate (kilo counts per second, kcps) versus time (days) illustrating stability of nanoparticles (formulation B, see Table 1) during long-term storage in water at room temperature, wherein particle stability was measured via DLS over 15 days.

FIG. 4 is a diagram showing particle size of 1 and 2 vol % perfluoro-n-pentane (PFP) immediately after emulsion formation in the nanoparticle pre-assembly solution (1:1 DMF:ACN, 1% TFA).

FIG. 5 is a graph showing relationship between PFP vaporization temperature (Tvap, degrees Centigrade, ° C.) and nanodroplet size (RH, hydrodynamic radius, nanometers, nm), modeled at three different reported surface tension values for PFP emulsions formulated with the BSA protein (open circles), PEO-PLA polymer (filled gray circles) or CTAB surfactant (filled black circles), where the dashed line indicates physiologic temperature (37° C.).

FIG. 6 is a graph of number percent versus particle size (nanometers, nm) illustrating particle size measurement of nanoparticles formulated under various peptide and PFP ratios, where each letter, A, B, C, D, and E, corresponds to different formulations shown in Table 1.

FIG. 7 shows the molecular structure of F_{F}-RGD.

FIG. 8 shows a schematic representative of peptide nanoparticles formulated using non-crosslinked N-pEM) of crosslinked (pEM) peptide monomers, 1D fibrils (1D0pEM) or 2D sheets (2D-pEM).

FIG. 9A shows a negatively stained TEM image of F_{F}-RGD nanofibrils; the insert shows a histogram of fibrillar diameter.

FIG. 9B shows the ThT fluorescence emission spectra in the presence of F_{F}-RGD in water (1D fibrils) or in 2.5% DMSO (2D sheets).

FIG. 9C shows the CD spectrum of F_{F}-RGD in water.

FIG. 10 shows a negatively stained TEM image of 2D F_{F}-RGD sheets.

FIG. 11 shows graphs indicating the size distribution of fluorous emulsions; N-pEM, pEM, 1D-pEM, and 2D-pEM (from left to right).

FIG. 12A shows a negatively stained TEM image of N-pEM.

FIG. 12B shows a negatively stained TEM image of pEM.

FIG. 12C shows a negatively stained TEM image of 1D-pEM; the yellow arrows denote fibrils adsorbed on the surface of the PFC droplet.

FIG. 12D shows a cryo-SEM image of 2D-pEM.

FIG. 13A shows a negatively stained TEM image of self-assembled F_{F}-RGD in water.

FIG. 13B shows a graph presenting the hydrodynamic diameter of self-assembled F_{F}-RGD in water.

FIG. 14 shows the molecular structure of non-fluorinated F-RGD.

FIG. 15A shows the CD spectrum of self-assembled F-RGD in water.

FIG. 15B shows a negatively stained TEM image of self-assembled F-RGD in water.

FIG. 16 shows photographs of pEMₚₕₑ (left) and 1D-pEMₚₕₑ (right) nanoparticle solutions prepared using the non-fluorinated F-RGD emulsifier where PFH was used as the fluorous liquid core; lack of opacity indicates an inability to form stable nanoparticles.

FIG. 17 shows representative photographs of four nanoparticle formations after assembly, demonstrating the variance in formulation opacity.

FIG. 18 shows bar graphs that present the percentage of nanoparticles vaporized (activated) following insonation at 37° C. under varying US intensities.

FIG. 19 shows optical images of 1D-pEM before and after insonation; inserts show a decrease in opacity of the 1D-pEM upon US-treatment, and formation of coalesced bubbles, indicative of emulsion vaporization.

FIG. 20 show photographs of 2D-pEM solution before (−US) and after (+US) acoustic sonication for 90 sec. at 2 W/cm² with a 50% duty cycle; appearance of bubbles and decrease in solution opacity in +US condition indicates nanoparticle vaporization.

FIG. 21A shows epifluorescence micrographs of Nile red-loaded 1D-pEM nanoparticles before exposure to US; insert shows accumulation of Nile red at the PFP-water interface where F_{F}-RGD fibrils are located.

FIG. 21B shows epifluorescence micrographs of Nile red-loaded 1D-pEM nanoparticles after exposure to US; arrows denote coalescence of 1D-pEMs that have recondensed after vaporization.

FIG. 22 shows fluorescence spectra of Nile red-loaded nanoparticles used for cell imaging and flow cytometry.

FIG. 23 shows bar graphs indicating the stability of nanoparticles after incubation either at room temperature or at 37° C. for 24 hours.

FIG. 24 shows bar graphs indicating the stability of nanoparticles at room temperature or at physiological temperature after incubation for 1-4 days.

FIG. 25 shows a bar graph indicating cell viability of RAW 264.7 macrophages measured after 24-hour incubation with four nanoparticles.

FIG. 26A shows epifluorescence micrographs of cells incubated with Nile red-loaded 1D-pEM for 6 hours; Middle: Nile red from nanoparticles; Bottom: DAPI from nuceli.

FIG. 26B shows epifluorescence micrographs of cells incubated with Nile red-loaded 1D-pEM for 24 hours; Middle: Nile red from nanoparticles; Bottom: DAPI from nuceli.

FIG. 27 shows fluorescent micrographs of RAW 264.7 macrophages after incubation with the indicated Nile red-loaded nanoparticles for 1 hour.

FIG. 28 shows fluorescent micrographs of RAW 264.7 macrophages after incubation with Nile red-loaded pEM.

FIG. 29 shows fluorescent micrographs of RAW 264.7 macrophages after incubation with Nile red-loaded 2D-pEM nanoparticles for 6 hours, 12 hours, and 24 hours.

FIG. 30 shows fluorescent micrographs of RAW 264.7 macrophages after incubation with Nile red-loaded nanoparticles for 48 hours.

FIG. 31 shows fluorescent micrographs of RAW 264.7 macrophages after incubation with Nile red-loaded nanoparticles for 72 hours.

FIG. 32 shows a graph presented side scattering intensity from macrophages incubated with Nile red-loaded nanoparticles for each time point.

FIG. 33 shows mean fluorescence intensity (MFI) of Nile red-loaded nanoparticles in macrophages analyzed by flow cytometry at each time point.

FIG. 34 shows mean fluorescence intensity (MFI) of Nile red-loaded nanoparticles in macrophages analyzed by flow cytometry at each time point.

FIG. 35 shows a bar graph presenting the viability of RAW 264.7 cells treated in the absence (US) or presence (1D-pEM, US) of insonated 1D-pEM nanoparticles.

FIG. 36 shows representative optical micrographs of 1D-pEM-RAW cells before US (−US) and after US (+US) acoustic insonation.

FIG. 37 is bar graph that compares pEM, 1D-pEM, and 2D-pEM nanoparticle B-mode echogenicity relative to blank media; region of interest (ROI) mean pixel intensity was measured from greyscale B-mode images.

FIG. 38 shows representative B-mode images of agar phantoms in the a) absence of nanoparticles or loaded with b) pEM, c) 1D-pEM, and d) 2D-pEM formulations.

FIG. 39A shows a representative B-mode image of an agar phantom loaded with 1D-pEM-RAW cells and insonated at acoustic pressures of P+=0.3 MPa, P−=0.3 MPa.

FIG. 39B shows a representative B-mode image of an agar phantom loaded with 1D-pEM-RAW cells and insonated at acoustic pressures of P+=1.1 MPa, P−=0.6 MPa.

FIG. 40 shows B-mode images of an agar phantom loaded with 1D-pEM-RAW cells dispersed in human blood.

FIG. 41 shows an experimental setup for measuring time-dependent B-mode contrast of 1D-pEM-RAW cells.

FIG. 42 shows representative B-mode images of 1D-pEM-RAW cells at varying exposure times; the rectangles define ROI used for quantification.

FIG. 43 show relative pixel intensity of 1D-pEM-RAW B-mode images over the 60 min insonation period.

FIG. 44 shows time-dependent B-mode images of a) cells only, and b, c) 1D-pEM-RAW cells; boxes represent the ROI for quantification, and time of exposure shown in the upper right corner of the image panel.

FIG. 45 shows (top) a schematic illustration of US macrophage tracking within porcine vasculature; (bottom) a photograph of ex vivo experimental setup for B-mode US imaging in the catheterized porcine coronary artery.

FIG. 46 shows representative time-dependent B-mode images of 1D-pEM-RAW cells during vessel perfusion; vessel walls are demarcated by dashed lines, dashed circles track individual 1D-pEM-RAW cells during flow, and white arrows represent flow direction.

FIG. 47 is a graph showing quantification of B-mode contrast for 1D-pEM-RAW cells compared to background controls.

FIG. 48 shows color doppler signals, superimposed on B-mode images collected during 1D-pEM-RAW cell vessel perfusion; white arrows represent flow direction.

FIG. 49 shows (left) B-mode and (right) Doppler US images of cells without nanoparticles in ex vivo setup; the dashed lines indicate vessel walls.

### DETAILED DESCRIPTION OF THE INVENTION

Compounds are described using standard nomenclature. Unless defined otherwise, all technical and scientific terms used herein have the same meaning as is commonly understood by one of skill in the art to which this invention belongs.

The terms “a” and “an” do not denote a limitation of quantity, but rather denote the presence of at least one of the referenced items. The term “or” means “and/or”. The terms “comprising,” “having,” “including,” and “containing” are to be construed as open-ended terms (i.e., meaning “including, but not limited to”).

Recitation of ranges of values are merely intended to serve as a shorthand method of referring individually to each separate value falling within the range, unless otherwise indicated herein, and each separate value is incorporated into the specification as if it were individually recited herein. The endpoints of all ranges are included within the range and independently combinable.

All methods described herein can be performed in a suitable order unless otherwise indicated herein or otherwise clearly contradicted by context. The use of any and all examples, or exemplary language (e.g., “such as”), is intended merely to better illustrate the invention and does not pose a limitation on the scope of the invention unless otherwise claimed. No language in the specification should be construed as indicating any non-claimed element as essential to the practice of the invention as used herein. Unless defined otherwise, technical and scientific terms used herein have the same meaning as is commonly understood by one of skill in the art of this disclosure.

Furthermore, the disclosure encompasses all variations, combinations, and permutations in which one or more limitations, elements, clauses, and descriptive terms from one or more of the listed claims are introduced into another claim. For example, any claim that is dependent on another claim can be modified to include one or more limitations found in any other claim that is dependent on the same-base claim. Where elements are presented as lists, e.g., in Markush group format, each subgroup of the elements is also disclosed, and any element(s) can be removed from the group.

All compounds are understood to include all possible isotopes of atoms occurring in the compounds. Isotopes include those atoms having the same atomic number but different mass numbers and encompass heavy isotopes and radioactive isotopes. By way of general example, and without limitation, isotopes of hydrogen include tritium and deuterium, and isotopes of carbon include ¹¹C, ¹³C, and ¹⁴C. Accordingly, the compounds disclosed herein may include heavy or radioactive isotopes in the structure of the compounds or as substituents attached thereto. Examples of useful heavy or radioactive isotopes include ¹⁸F, ¹⁵N, ¹⁸O, ⁷⁶Br, ¹²⁵I and ¹³¹I.

The opened ended term “comprising” includes the intermediate and closed terms “consisting essentially of and “consisting of.”

A dash (“-”) that is not between two letters or symbols is used to indicate a point of attachment for a substituent.

“Pharmaceutical compositions” means compositions including at least one active agent, such as a compound or salt of Formula 3, and at least one other substance, such as a carrier. Pharmaceutical compositions meet the U.S. FDA's GMP (good manufacturing practice) standards for human or non-human drugs.

A “patient” means a human or non-human animal in need of medical treatment. Medical treatment can include treatment of an existing condition, such as a disease or disorder or diagnostic treatment. In some embodiments the patient is a human patient.

“Providing” means giving, administering, selling, distributing, transferring (for profit or not), manufacturing, compounding, or dispensing.

“Treatment” or “treating” means providing an active compound to a patient in an amount sufficient to measurably reduce any disease symptom, slow disease progression or cause disease regression. In certain embodiments treatment of the disease may be commenced before the patient presents symptoms of the disease.

A “therapeutically active agent” means a compound which can be used for diagnosis or treatments of a disease. The compounds can be small molecules, peptides, proteins, or other kinds of molecules.

A significant change is any detectable change that is statistically significant in a standard parametric test of statistical significance such as Student's T-test, where p<0.05.

The present invention is directed to novel nanoparticles prepared through the templated assembly of amphiphilic peptides at a fluorous-liquid interface containing releasable cargo-fluorine-containing cargo solubilizing agent complexes.

A “nanoparticle” refers to a structured entity capable of incorporating a variety of functional components. Nanoparticles, along with, but not limited to, the terms “nanoparticle”, “nanoparticle”, “nanoemulsion”, “emulsion”, and “nanofibril”, denote an arrangement of amphiphilic peptides forming a corona around a perfluorocarbon liquid core. This core is capable of undergoing a phase transition upon ultrasound stimulation, thus facilitating the targeted release of encapsulated cargo. The term nanoparticle encompasses a spectrum of such formulations, characterized by their molecular architecture that includes a lipid-like core surrounded by a shell of peptides, where these peptides may possess properties such as hydrophilicity, hydrophobicity, and specific binding functionalities.

Nanoparticles according to embodiments of the present invention have a perfluorocarbon liquid core that phase transitions into a gaseous state upon ultrasound application. When positioned at the cell surface, ultrasound activation serves to deliver cargo encapsulated within the nanoparticles into the cytoplasm directly. Thus, proteins, peptides, nucleic acids, small molecule compounds, and other materials, can be encapsulated and directly delivered to the cytoplasm of cells without loss of function. This ultrasound-mediated delivery is ideal for therapeutics due to its spatial and temporal precision.

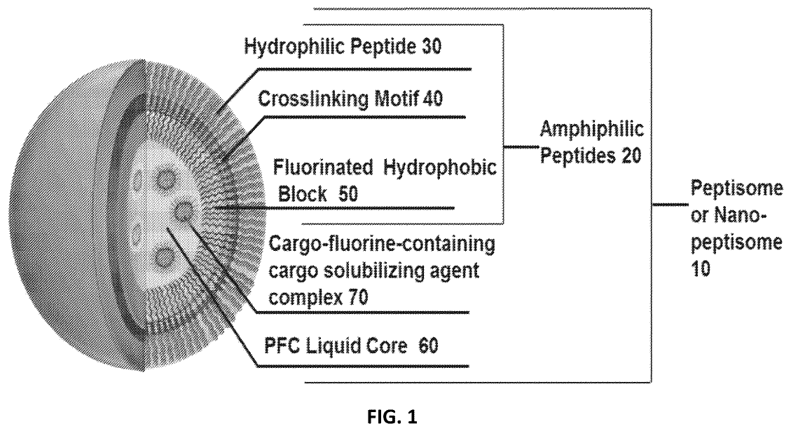

A typical nanoparticles shown at 10 in FIG. 1 including a plurality of amphiphilic peptides 20, wherein the amphiphilic peptides each include a hydrophilic peptide 30, a crosslinking motif 40, and a fluorinated hydrophobic block 50. The fluorinated hydrophobic block of the amphiphilic peptides promotes peptide assembly at the surface of the perfluorocarbon (PFC) liquid core 60, which contains the cargo-fluorine-containing cargo solubilizing agent complexes 70.

In some embodiments, the cargo is not dispersed in the perfluorocarbon liquid core 60 but is instead associated with or bound to the surface of the external amphiphilic peptide 20 molecules through various binding mechanisms, including but not limited to electrostatic interactions, ionic interactions, or other specific affinity-based interactions. In some embodiments, the amphiphilic peptide 20 molecules may contain charged amino acid residues that engage in electrostatic interactions with oppositely charged domains on the cargo, thereby enhancing the stability and specificity of their surface attachment.

Nanoparticles having a diameter of from about 250 nm to about 5 microns may be produced. An average diameter of the nanoparticles according to embodiments may be from about 1 to about 5 microns, for example, about 1 to about 4 microns, about 1 to about 3 microns, or about 1 to about 2 microns, but is not limited thereto. In an embodiment, the nanoparticles have an average diameter in the range of about 300 nanometers to 1200 nanometers, about 250 nanometers to about 1000 nanometers, for example, 250 to about 750 nanometers, but is not limited thereto.

The nanoparticles contain a perfluorocarbon liquid core that allows for activation of the nanoparticles upon application of ultrasound (US) and delivery of a cargo present in the perfluorocarbon liquid core. The term “activation” as used herein to refer to activation of nanoparticles upon application of ultrasound refers to phase transition of a perfluorocarbon liquid core into a gaseous state due to ultrasound application.

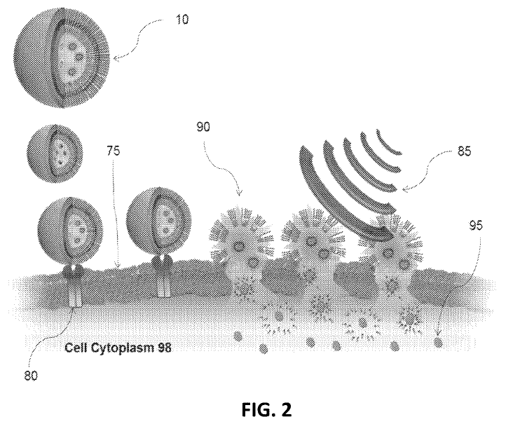

As shown diagrammatically in FIG. 2, nanoparticle 10 binds to a receptor 80 disposed in or on a cell membrane 75 via specific interaction with the hydrophilic peptide 30 of the amphiphilic peptides of nanoparticle 10. Application of ultrasound 85 causes acoustic vaporization of the perfluorocarbon liquid core of the nanoparticles and leads to the formation of a gaseous core that ultimately swells and ruptures 90 the nanoparticles. Subsequent bubble captivation produces a high intensity pressure wave that, when generated at the surface of a cell, transiently permeabilizes the plasma membrane 75, and simultaneously ejects cargo 95 encapsulated in the nanoparticle into the cell cytoplasm 98. Thus, US-sensitive nanoparticles represent a spatially and temporally controlled delivery modality that, as described herein, can deliver a cargo, such as biomacromolecules, directly into the cytoplasm of cells, thereby avoiding endosomal uptake and degradation of the bioactive payload.

Key to the assembly of these nanoparticles is a de novo designed amphiphilic peptide, capable of assembling at the surface of a perfluorocarbon liquid.

As noted above, amphiphilic peptides included in nanoparticles according to embodiments each include a fluorinated hydrophobic block (HB), a crosslinking motif, and a hydrophilic peptide.

Thus, in one embodiment, an amphiphilic peptide represented by Formula (I) is provided:

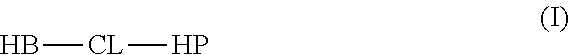

wherein HB is a fluorinated hydrophobic polymer; CL is a cross-linking motif; and HP is a hydrophilic amino acid sequence.

As used herein, the term “amphiphilic peptide” refers to a molecule including a fluorinated hydrophobic polymer; a cross-linking motif; and a hydrophilic amino acid sequence, wherein the amphiphilic peptide has a molecular weight in the range of about: 2000-5000 daltons, wherein the amphiphilic peptide includes at least five amino acid residues, and a total number of no more than 30 amino acid residues, wherein at least two of the amino acid residues are consecutively linked to each other in a chain by a peptide bond.

As used herein, the term “fluorinated hydrophobic polymer” refers to a covalently linked chain of monomer residues terming a fluorinated hydrophobic homopolymer or copolymer. The monomeric units which form the fluorinated hydrophobic polymer may each be fluorinated according to embodiments, or some, or one, of the monomeric units is fluorinated such that at least one or more of the monomer residues of the fluorinated hydrophobic polymer is fluorinated,

According to embodiments, the amphiphilic peptide does not include lipids.

According to embodiments, the fluorinated hydrophobic polymer includes a hydrophobic amino acid sequence wherein the amino acids of the hydrophobic amino acid sequence have non-polar side chains, wherein the non-polar side chains do not include a group capable of forming a hydrogen bond with molecules of water; and wherein at least one of the amino acids of the hydrophobic amino acid sequence is fluorinated.

According to embodiments, the fluorinated hydrophobic polymer includes one or more synthetic non-amino acid monomeric units wherein at least one of the monomeric units is fluorinated such that at least one of the monomer residues of the fluorinated hydrophobic polymer is fluorinated. Non-limiting examples of synthetic monomeric units which can be fluorinated and reacted to form a fluorinated hydrophobic polymer include methyl methacrylate; lactic acid, glycolic acid and olefins such as ethylene, propylene, styrene.

As used herein, the term “hydrophobic amino acid sequence” refers to a hydrophobic polymer, a sequence of hydrophobic amino acids having non-polar side chains, wherein the non-polar side chains do not include a group capable of forming a hydrogen bond with molecules of water, or a combination of a hydrophobic polymer and a sequence of hydrophobic amino acids having non-polar side chains. Hydrophobic ammo acids may be naturally occurring; or non-natural (artificially produced). Examples of the naturally occurring hydrophobic amino acids include, but are not limited to, alanine, valine, leucine, isoleucine, proline, phenylalanine, tryptophan, cysteine, and methionine. Examples of the non-natural hydrophobic amino acids may include D amino acids, as well as specific non-natural amino acids such as selenocysteine, pyrrolysine, and the like.

In the amphiphilic peptide, the fluorinated hydrophobic amino acid sequence may include one to ten fluorinated hydrophobic amino acids consecutively connected by peptide bonds, which may be unsubstituted or substituted with a substituent selected from —F, —Cl, —Br, —I, a C₁-C₃₀ alkyl group, a C₂-C₃₀ alkenyl group, a C₂-C₃₀ alkynyl group, a C₃-C₃₀ cycloalkyl group, a C₃-C₃₀ cycloalkenyl group, a C₆-C₃₀ aryl group, a C₇-C₃₀ arylalkyl group, but are not limited thereto. For example, the fluorinated hydrophobic amino acid sequence may include one, two, three, four, five, six seven, eight, nine or ten fluorinated hydrophobic amino acids consecutively connected by peptide bonds. Fluorinated hydrophobic amino acids include, for example, fluorinated alanine, fluorinated saline, fluorinated leucine, fluorinated isoleucine, fluorinated proline, fluorinated phenylalanine, fluorinated tryptophan, fluorinated cysteine, fluorinated methionine, fluorinated selenocysteine, and fluorinated pyrrolysine. The fluorinated hydrophobic amino acids can be D or L amino acids and can be fluorinated at any suitable position, typically replacing a hydrogen atom. In an embodiment, the fluorinated hydrophobic amino acid sequence may include pentafluoro-phenylalanine (2,3,4,5,6-pentafluoro-L-phenylalanine and/or 2,3,4,5,6-pentafluoro-D-phenylalanine) at a terminal thereof. In another embodiment, the fluorinated hydrophobic amino acid sequence may include one to ten, such as one, two, three, four, five, six, seven, eight, nine or ten consecutively connected pentafluoro-phenylalanine residues at a terminal thereof.

A combination of a hydrophobic polymer and a sequence of hydrophobic amino acids having non-polar side chains can be included in the fluorinated hydrophobic polymer wherein at least one of the monomer residues of the fluorinated hydrophobic polymer is fluorinated and/or at least one of the amino acid residues is fluorinated.

As used herein, the term “hydrophilic amino acid sequence” refers to a sequence of hydrophilic amino acids consecutively connected by peptide bonds, wherein the hydrophilic amino acids have a polar side chain, wherein the polar side chain includes a group capable of forming a hydrogen bond with molecules of water. Hydrophilic amino acids may be naturally occurring or non-natural and can be D or L amino acids. Examples of the naturally-occurring hydrophilic amino acids include, but are not limited to, senile, threonine, asparagine, glutamine, histidine and tyrosine.

Examples of the non-natural hydrophilic amino acids include amino acids having various heterocyclic groups as a part of the side chain.

In the amphiphilic peptide, the hydrophilic amino acid sequence HP may include three to fifteen hydrophilic amino acids consecutively connected by peptide bonds. For example, the hydrophilic amino acid sequence HP may include one, two, three, four, five, six, seven, eight, nine, ten, eleven, twelve, thirteen, fourteen, or fifteen hydrophilic amino acids consecutively connected by peptide bonds.

In one embodiment, an amphiphilic peptide represented by Formula (II) is provided:

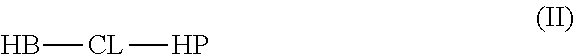

wherein HB is a fluorinated hydrophobic amino acid sequence; CL is a cross-linking motif; and HP is a hydrophilic amino acid sequence.

In one embodiment, an amphiphilic peptide represented by Formula (III) is provided:

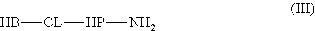

wherein HB is a fluorinated hydrophobic amino acid sequence; CL is a cross-linking motif; and HP is a C-terminally amidated hydrophilic amino acid sequence.

In one embodiment, the hydrophilic amino acid sequence includes a targeting agent that interacts with a targeted component of a target cell. The targeted component is at least partially external to the target cell and interaction of the targeting agent and targeted component of the target cells server to bring nanoparticles into proximity with the target cell into which the cargo is to be delivered. The target cells can be cells of any organism, such as, but not limited to, a mammal, bird, fish, or bacterial cell. According to embodiments, the target cell is a human cell or a bacterial cell within a human body.

According to embodiments, the targeting agent includes a minimal targeting motif peptide and optionally includes one or more hydrophilic amino acids attached to the N-terminus or C-terminus of the minimal targeting motif peptide by peptide bonds. Typically, amino acids of the targeting motif peptide are L-amino acids but these may include one or more D-amino acids so long as the targeting motif still correctly mediates binding with the targeted component. The one or more hydrophilic amino acids attached to the N-terminus or C-terminus of the minimal targeting motif peptide by peptide bonds can be D or L amino acids.

According to embodiments, the targeting agent includes a minimal targeting motif peptide selected from: HGK, RGD, KAR, RSR, KAA, RGRR (SEQ ID NO:1), RGRS (SEQ ID NO:2), YQLDV (SEQ ID NO:3), EYQ, RPM, PSP, VGVA (SEQ ID NO:4), NGR, CRKRLDRNC (SEQ ID NO 43) which binds to the IL-4 receptor on atherosclerotic plaques; EFEEFEIDEEEK (SEQ ID NO:44) which binds to thrombin in blood clots; and/or DFEEIPEEYLQ (SEQ ID NO:45) which binds to thrombin in blood clots, and optionally includes one or more hydrophilic amino acids attached to the N-terminus or C-terminus of the minimal targeting motif peptide by peptide bonds.

For example, the hydrophilic amino acid sequence HP may include the amino acid sequence KGRGD (SEQ ID NO:35) as a targeting agent, wherein K is lysine, Cr is glycine, R is arginine, and O is aspartic acid, which includes minimal targeting motif RGD capable of specific binding to integrins and two hydrophilic amino acids.

Minimal targeting motif RGD alone or with additional hydrophilic amino acids along with the binding affinity to αVβ integrin (IC₅₀ conc. in nM at which 50% of receptor is bound by ligand): RGD (89±12), RGDS (45±5, SEQ ID NO:36), GRGD (55±7, SEQ ID NO: 37), GRGDS 28±3, SEQ ID NO:38), GRGDSP (13.7±0.3, SEQ ID NO: 39), GRGDSPK (12.2±0.1, SEQ ID NO:40), GRGDNP (45±12, SEQ ID NO:41), and GRGDTP (28±5, SEQ ID NO:42x). Thus, according to embodiments, the hydrophilic amino acid sequence HP includes, RGD, KGRGD (SEQ ID NO:35), RGDS (SEQ ID NO:36), GRGD (SEQ ID NO:37), GRGDS (SEQ ID NO:38), GRGDSP (SEQ ID NO:39), GRGDSPK (SEQ ID NO:40), GRGDNP (SEQ ID NO:41), or GRGDTP (SEQ ID NO:42).

Various targeting agents and motifs are described in US Patent Publication No 20200197307, which is hereby incorporated by reference in its entirety.

As used herein, the phrase “cross-linking motif” refers to an amino acid sequence that includes, at any position in the sequence, at least two amino acid residues each capable of cross-linking with a corresponding amino acid residue capable of cross-linking and present in another amphiphilic peptide.

The at least two amino acid residues capable of cross-linking with a corresponding amino acid residue in the crosslinking motif of another amphiphilic peptide can be a naturally occurring amino acids and/or a non-naturally occurring amino acids.

Naturally occurring amino acids capable of crosslinking with a corresponding amino acid residue include cysteine.

An amino acid may be functionalized to such that it is a non-naturally occurring amino acid to provide the ability to bind to a naturally occurring or non-naturally occurring amino acid in the crosslinking motif of another amphiphilic peptide.

In exemplary embodiments, the cross-linking motif may include cross linking moieties such as sulfhydryl crosslinkers, UV cross-linkers, aza-benzenes, photosensitive crosslinkers such as asides or benzophenones, nitriles, pH sensitive cross-linkers, or enzymatic cross-linkers.

In an embodiment, the cross-linking motif may include cysteine, and may optionally further include glycine. For example, in one preferred embodiment, the cross-linking motif CL may include GGGCCGG (SEQ ID NO:46), wherein G is glycine and C is cysteine. The crosslinking motif CL may comprise from 1 to about 10 amino acid residues.

In the nanoparticles, the amphiphilic peptide molecules are oriented in such a way that groups HB of the peptide are located at a surface of the perfluorocarbon of the perfluorocarbon liquid core, wherein the amphiphilic peptide molecules are bonded intramolecularly. For example, when the cross-linking motif of the amphiphilic peptide includes a cysteine residue, the amphiphilic peptide molecules may be cross-linked via disulfide cross-linking groups (—S—S—). When the cross-linking motif of the amphiphilic peptide includes two or more cysteine residues, the two or more cysteine residues may be intramolecularly connected via disulfide cross-lining: groups (—S—S—).

A degree of cross-linking of the amphiphilic peptide molecules may be about 50% or greater, for example, about 55% or greater, about 60% or greater, about 65% or greater, about 70% or greater, about 75% or greater, about 80% or greater, about 85% or greater, about 90% or greater, or about 95% or greater. The degree of crosslinking may be measured using a colorimetric disulfide formation assay (described in detail below).

The amphiphilic peptide, according to an embodiment, may include 5 to 30 amino acids and has a molecular weight in the range of about 2000-5000 daltons. For example, the amphiphilic peptide may include 5, 6, 7, 8, 9, 10, 11, 12, 13, 14, 15, 16, 17, 18, 19, 20, 21, 22, 23, 24, 25, 26, 27, 28, 29 or 30 amino acids wherein the amphiphilic peptide has a molecular weight in the range of about 2000-5000 daltons.
in an embodiment, the amphiphilic peptide has Formula (IV):

(IV)
(SEQ ID NO: 47)
F_{F}F_{F}F_{F}GGGCCGGKGRGD

wherein F_{F} is pentafluoro-phenylalanine (2,3,4,5,6-pentafluoro-L-phenylalanine), G is glycine, C is cysteine, K is lysine, G is glycine, R is arginine, and D is aspartic acid. In Formula (IV), FFFFFF is a hydrophobic block HB, GGGCCGG (SEQ ID NO:46) is a cross-linking motif CL, and KGRGD-NH₂ (SEQ ID NO:48) is a C-terminally amidated hydrophilic amino acid sequence HP, which contains targeting motif RGD.

In an embodiment, the amphiphilic peptide has Formula (V):

(V)
(SEQ ID NO: 49)
F_{F}F_{F}F_{F}GGGCCGGKGRGD-NH₂

wherein F_{F} is pentafluoro-phenylalanine, G is glycine, C is cysteine, K lysine, G is glycine, R is arginine, and D is aspartic acid, and the amphiphilic peptide is C-terminally amidated. In Formula (IV), FFFFFF is a hydrophobic block HB, GGGCCGG (SEQ ID NO:46) is a cross-linking motif CL, and KGRGD-NH₂ (SEQ ID NO:48) is a C-terminally amidated hydrophilic amino acid sequence HP, which contains targeting motif RGD.

Amphiphilic peptides can be synthesized using techniques known to one of ordinary skill in the art, such as, but not limited to, solid-phase synthesis, recombinant methodologies, polymerization, and conjugation methods.

Advantageously, amphiphilic peptides according to embodiments of the present invention may include highly fluorinated amino acid residues and such sequences may be chemically synthesized in high yield and purity using standard solid-phase techniques known to one of ordinary skill in the art.

The amphiphilic peptides of all of Formulas (I), (II), (III), (IV), and (V) are capable of assembling at the surface of a perfluorocarbon liquid and assemble into a layer to form a nanoparticle.

Amphiphilic peptides (IV) and (V) contain three pentafluoro-phenylalanine (F_{F}) residues at the N-terminus which promote interpolation and assembly of the peptide at the perfluorocarbon liquid interface. C-terminal to this fluorous domain is a cysteine-containing crosslinking motif (GGGCCGG, SEQ ID NO:46) designed to undergo disulfide cross-linking to an adjacent amphiphilic peptide in order to stabilize the peptide corona after templated assembly. Incorporation of a bioactive hydrophilic sequence at the peptide's C-terminus ultimately leads to its multivalent display at the surface of the assembled particle. The amphiphilic peptides of Formulas (IV) and (V) include the sequence KGRGD (SEQ ID NO:35) to enable. cell-surface localization of nanoparticles including the amphiphilic peptides of Formulas (IV) and (V) mediated by binding of the targeting motif RGD with extracellular integrins.

As noted above, the nanoparticles contain a perfluorocarbon (WC) liquid core that allows for activation of the particle upon application of ultrasound (US) and delivery of a cargo present in the perfluorocarbon liquid core.

As used herein, the term “perfluorocarbon” refers to a hydrocarbon in which, all or a substantial portion of hydrogen atoms in bonds are replaced with fluorine atoms, producing C—F bonds, The degree of replacement of hydrogen atoms with fluorine atoms may vary, and may be 100%, 99% or greater. 98% or greater, 97% or greater, 96% or greater, 95% or greater, 90% or greater, 85% or greater, 80% or greater, 75% or greater, or 70% or greater. In another embodiment, the degree of replacement of hydrogen atoms with fluorine atoms may be 100%, 99.9% or greater, 99.8% or greater, 99.7% or greater, 99.6% or greater, 99.5% or greater; 99.4% or greater, 99.3% or greater, 99.2% or greater, or 99.1% or greater. The perfluorocarbon liquid may be a perfluorobutane, a perfluoropentane, a perfluorohexane, octafluoropropane, but is not limited thereto. The perfluorocarbon liquid may be a perfluoropentane, for example, perfluoro-n-pentane (PFP) or perfluoro-iso-pentane. The perfluorocarbon liquid may be a perfluorohexane, for example, perfluoro-n-hexane (PFH), perfluoro-iso-hexane, or perfluoro-see-hexane. As used herein, the term “perfluorocarbon liquid” generally refers to a perfluorocarbon as defined above, which is present in a liquid state at ambient temperature of about 25° C. The perfluorocarbon liquid may have a boiling point of about 45° C., or lower, for example, about 40° C. or lower, about 35° C. or lower, or about 30° C. or lower. While not wishing to be bound to any theory, it is understood that the higher the boiling point of the perfluorocarbon liquid, the greater ultrasound intensity should be utilized to acoustically vaporize the perfluorocarbon liquid core of the nanoparticles which leads to the formation of a gaseous core that ultimately swells and ruptures the nanoparticles. Accordingly, when the boiling point of the perfluorocarbon liquid is too high, for example, greater than 45° C., cells may be damaged by the application of the ultrasound.

On the other hand, the intensity of the ultrasound should be sufficient to release the cargo from the nanoparticles into the cells. To ensure, however, that no cell damage occurs, the intensity of the ultrasound should not be greater than 1.0 watts per square centimeter (W/cm²), and its mechanical index (MI) should be maintained below 1.9. Ultrasound systems for in vitro and in vivo application are commercially available, such as Toshiba Medical Systems Aplio500 and GE Healthcare Logiq E9, and these and other such systems can be used according to the manufacturers specifications to administer ultrasound to a patient, or to isolated cells to image nanoparticles to targeted cells or regions such as atherosclerotic plaques or blood clots.

The perfluorocarbon liquid core may further include a bioimaging agent, for example, a photoacoustic dye (such as indocyanine green “ICG”, Cyanine 7 “Cy7”, or dimethyl{4-[1,5,5-tris(4-dimethylaminophenyl)-2,4-pentadienylidene]-2,5=cyclohexadiene-1-ylidne}ammonium perchlorate “IR800”), a fluorescent dye or protein (such as green fluorescent protein “GFP”, fluorescein, rhodamine, a cyanine dye), and a magnetic resonance imaging “MRI contrast agent (such as iron oxide or gadolinium), a radiotracer, but is not limited thereto.

According to embodiments, the perfluorocarbon liquid core includes about 1×10³ to about 5×10⁹ molecules, such as 1×10⁴ to about 5×10⁸ molecules of the active agent, such as 1×10⁵ to about 5×10⁷ molecules of the active agent, such as 1×10⁶ to about 5×10⁶ molecules of the active agent, and may include more, or less, of the active agent.

According to embodiments, a cargo to be delivered to the interior of a cell via the nanoparticles is contacted with is fluorine-containing cargo solubilizing agent to aid in miscibility with the perfluorocarbon liquid core. The fluorine-containing cargo solubilizing agent may be, for example, a perfluoroalkyl, a polyfluoroalkyl, a perfluorinated alkyl acid, a polyfluorinated alkyl acid, a perfluorinated aromatic compound, a polyfluorinated aromatic compound, any of which may be further substituted or unsubstituted, or a mixture of any two or more thereof. The fluorine-containing cargo solubilizing agent. may be, for example, perfluorooctane (CF₃(CF₂)₆CF₃), perfluoroteradecane (CF₃(CF₂)₁₂CF₃), trifluoroacetic acid (CF₃COOH), pentafluoropropionic acid (CF₃(CF₂)COOH), perfluorotetradecanoic acid (CF₃(CF₂)₁₂COOH), perfluorooctadecanoic acid (CF₃(CF₂)₁₆COOH), perfluorocyclohexanecarboxylic acid ((CF₂)₅CFCOOH), pentafluorophenol (2,3,4,5,6-pentafluorophenol, C₆F₅OH), pentafluorobenzaldehyde (2,3,4,5,6-pentafluorobenzaldehyde, C₆F₅CHO), or Fmoc-pnetafluorophenylalanine ((CF)₅CCH₂C(NH-Fmoc)COOH, Fmoc-pentafluoro-L-phenylalanine and/or Fmoc-pentafluoro-D-phenylalanine, or a mixture of any two or more thereof, but is not limited thereto. In an embodiment, the fluorine-containing cargo solubilizing agent may be perfluorononanoic acid.

In another embodiment, a composition including the above nanoparticle is provided that may be used for therapeutic or diagnostic use. The composition may include a pharmaceutically acceptable excipient for example, a vehicle, an adjuvant, a carrier or a diluent, that are well-known to those who are skilled in the art and are readily available to the public. Typically, the pharmaceutically acceptable carrier is one that is chemically inert to the pharmaceutically active agents and one that has no detrimental side effects or toxicity under the conditions of use.

The compositions may be administered as oral, sublingual, transdermal, subcutaneous, topical, absorption through epithelial or mucocutaneous linings, intravenous, intranasal, intraarterial, intramuscular, intratumoral, peritumoral, interperitoneal, intrathecal, rectal, vaginal, or aerosol formulations. In some aspects, the pharmaceutical composition is administered orally or intravenously. One preferred method of administration is through an intravenous injection.

In still another embodiment, a method of preparing the nanoparticle is disclosed. According to the method, a composition including a therapeutically active agent, an amphiphilic peptide represented by any of the above Formulas (I), (II), (III), (IV), or (V), and a perfluorocarbon liquid is provided. The composition is then contacted with water to provide an intermediate assembly including a perfluorocarbon liquid core containing the perfluorocarbon liquid and the therapeutically active agent dispersed in the perfluorocarbon liquid, and a plurality of amphiphilic peptides surrounding the perfluorocarbon liquid core. In the intermediate assembly, the amphiphilic peptides are oriented in such a way that the groups HB are interpolated into the perfluorocarbon liquid of the perfluorocarbon liquid core, while the groups HP extend away from the surface of the perfluorocarbon liquid core and away from the core of the perfluorocarbon liquid core. The amphiphilic peptide molecules of the intermediate assembly are subsequently cross-linked to form the nanoparticles.

The water may have a temperature of 10° C. or lower, for example, 9° C. or lower, 8° C. or lower, 7° C. or lower, 6° C. or lower, 5° C. or lower, 4° C. or lower, 3° C. or lower, 2° C. or lower, or 1° C. or lower. In an embodiment, the water may be ice-cold water. While not wishing to be bound to any theory, it is understood that when cold water is slowly added to an organic emulsion of amphiphilic peptides and perfluorocarbon liquid, spontaneous assembly of the amphiphilic peptides at the surface of the per-fluorocarbon liquid core takes place. This mild procedure also eliminates the need for aggressive synthetic methods commonly used to prepare stimuli-responsive particles, which can lead to degradation of the encapsulated therapeutically active agent.

According to embodiments of methods of making nanoparticles, a cargo to be delivered to the interior of a cell via the nanoparticles is contacted with a fluorine-containing cargo solubilizing agent to aid in miscibility with the perfluorocarbon liquid core. The fluorine-containing cargo solubilizing agent may be, for example, a perfluoroalkyl, a polyfluoroalkyl, a perfluorinated alkyl acid, a polyfluorinated alkyl acid, a perfluorinated aromatic compound, a polyfluorinated aromatic compound, any of which may be further substituted or substituted, or a mixture of any two or more thereof. The fluorine-containing cargo solubilizing agent may be, for example, perfluorooctane (CF₃(CF₂)₆CF₃, perfluoroteradecane (CF₃(CF₂)₁₂CF₃), trifluoroacetic acid (CF₃COOH), pentafluoropropionic acid (CF₃(CF₂)COOH), perfluoropentanoic acid (CF₃(CF₂)₃COOH), perfluorononanoic acid (CF₃(CF₂)₇COOH), perfluorotetradecanoic acid (CF₃(CF₂)₁₂COOH), perfluorooctadecanoic acid (CF₃(CF₂)₁₆COOH), perfluorocyclohexanecarboylic acid ((CF₂)₅CFCOOH), pentafluorophenol (2,3,4,5,6-pentafluorophenol, C₆F₅OH), pentafluorobenzaldehyde (2,3,4,5,6-pentafluorobenzaldehyde, C₆F₅CHO), or Fmoc-pnetafluorophenylalanine ((CF)₅CCH₂C(NH-Fmoc)COOH, Fmoc-pentafluoro-L-phenylalanine and/or Fmoc-pentafluoro-D-phenylalanine, or a mixture of any two or more thereof, but is not limited thereto. In an embodiment, the fluorine-containing cargo solubilizing agent may be perfluorononanoic acid.

According to embodiments of methods of making nanoparticles, the fluorine-containing cargo solubilizing agent, the perfluorocarbon liquid, and the cargo are mixed together and the amphiphilic peptides represented by any of the above Formulas (I), (II), (III), (IV), or (V), are added, forming a composition. The composition is then contacted with water to provide an intermediate assembly including a pet-fluorocarbon core containing the perfluorocarbon liquid and the therapeutically active agent dispersed in the perfluorocarbon liquid, and a plurality of amphiphilic peptides surrounding the perfluorocarbon liquid core, In the intermediate assembly, the amphiphilic peptides are oriented in such a way that the groups HB are interpolated into the perfluorocarbon liquid of the perfluorocarbon liquid core, while the groups HP extend away from the surface of the perfluorocarbon liquid cote and away from the core of the perfluorocarbon liquid core. The amphiphilic peptide molecules of the intermediate assembly are subsequently cross-linked to form the nanoparticles.

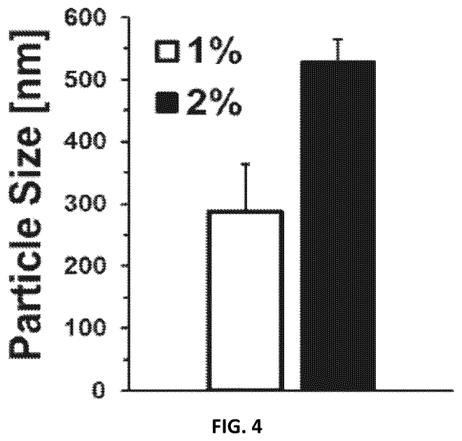

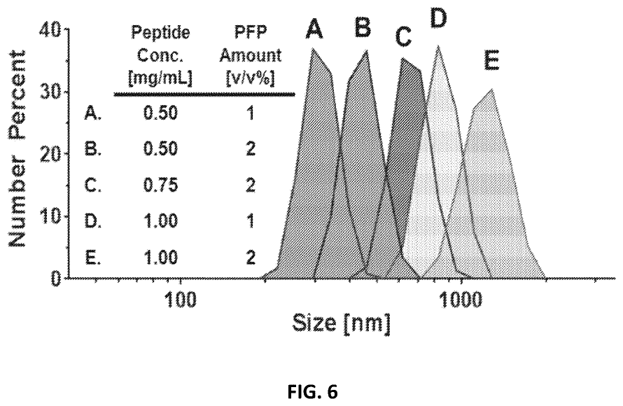

According to embodiments, the nanoparticles have an average diameter in the range from about 1 micron to about 5 microns. In other embodiments, the nanoparticles have an average diameter in the range from about 250 nanometers to about 1000 milometers. According to embodiments, the nanoparticles have an average diameter in the range from about 250 nanometers about 750 nanometers, The size of nanoparticles can be controlled by varying the volume percent (vol %) of the perfluorocarbon liquid and/or the concentration of amphiphilic peptide in the composition when making the nanoparticles, see for example FIG. 4 and FIG. 6. In general, the volume percent of the perfluorocarbon liquid can be increased to increase the average diameter of the nanoparticles, but this increase in average diameter is limited if the concentration of amphiphilic peptide is not also increased. According to embodiments, the volume percent of the perfluorocarbon liquid can be increased to increase the average diameter of the nanoparticles with a standard amount of amphiphilic peptides, along with simultaneous additional preparations in which the amount of amphiphilic peptides is varied to obtain a population of nanoparticles with a desired average diameter.

During preparation, the cross-linking may be performed during a dialysis of the intermediate assembly. The dialysis may be conducted in an aqueous solution including dimethylsulfoxide or any other organic solvent capable of oxidizing and cross-linking filial groups of cysteine amino acids. For example, the dialysis mays be carried out in an aqueous solution of dimethylsulfoxide (DMSO) at any concentration. In an embodiment, the dialysis can be carried out in a 2.5% solution of DMSO in water. This mild cross-linking procedure also eliminates the need for aggressive synthetic methods commonly used to prepare stimuli-responsive particles, which can lead to degradation of the encapsulated cargo of the therapeutically active agent.

The degree of cross-linking of the amphiphilic peptide molecules is about 60% or greater, for example, about 65% or greater, about 70% or greater, about 75% or greater, about 80% or greater, about 85% or greater, about 90% or greater, or about 95% or greater. The degree of cross-linking of the amphiphilic peptides can be determined by a colorimetric disulfide formation assay.

According to embodiments, nanoparticles are utilized in ultrasound imaging methods. Nanoparticles having an average diameter of >1 micron provide good acoustic contrast and thus are useful as imaging agents. Such nanoparticles are too large to leave blood vessels and therefore are particularly useful in vascular applications, such as targeted imaging for diagnosis and/or targeted, treatment of atherosclerotic plaques or blood clots,

In contrast, nanoparticles<750 nm in diameter can leave blood vessels in diseased tissues and other tissues and distribute to cells, thereby allowing, for cargo delivery, including targeted cargo delivery, such as delivery of drugs and biologics including, but not limited to, small molecules, proteins and nucleic acids.

For imaging, two modalities of ultrasound imaging can be used. B-mode ultrasound imaging allows viewing of stable nanoparticles that have cores vaporized under low intensity ultrasound to form microbubbles—but have not yet collapsed or lysed. B-mode ultrasound imaging allows a user to view and guide the nanoparticles in space using the ultrasound pressure wave. Doppler imaging can be used and allows viewing of changes in frequency that occur when the nanoparticles collapse due to application of ultrasound.

The term “low intensity” is used to refer to ultrasound at acoustic pressures that allow the core of the nanoparticles to oscillate as bubbles but not collapse. The term “high intensity” is used to refer to ultrasound at acoustic pressures that cause bubble cavitation of the nanoparticle cores. The exact threshold defining where low intensity stops, and high intensity starts will depend on the nature of the peptide shell and size of the nanoparticles. In general, application of ultrasound to a patient is at an ultrasound intensity of no higher than 1.9 MI. For example, the 500 nm nanoparticles wherein the amphiphilic peptides have the sequence F_{F}F_{F}F_{F}GGGCCGGKGRGD-NH₂ (SEQ ID NO:49), stably oscillate as bubbles below 0.4 MI (mechanical index, measure of ultrasound intensity), and collapse at ultrasound pressures above this threshold.

In some embodiments, nanoparticles can be formed with varying acoustic properties. For example, adding water either before or after amphiphilic peptides contact the perfluorocarbon liquid, and then either cross-linking or not cross-linking the amphiphilic peptides, results in four distinct morphologies of nanoparticles. In some embodiments, when water is added to plurality of peptide-based nanoparticles after their formation and the amphiphilic peptides of each peptide-based nanoparticle are crosslinked to each other, the resulting nanoparticles comprise a crosslinked unimolecular monolayer morphology. In some embodiments, when water is added to plurality of peptide-based nanoparticles after their formation and the amphiphilic peptides of each peptide-based nanoparticle are not crosslinked to each other, the resulting nanoparticles comprise a non-crosslinked unimolecular monolayer morphology. In some embodiments, when water is added to the plurality of amphiphilic peptides prior to their contact with the perfluorocarbon liquid and the amphiphilic peptides of each peptide-based nanoparticle are crosslinked to each other, the resulting nanoparticles comprise a 2D sheet morphology. In some embodiments, when water is added to the plurality of amphiphilic peptides prior to their contact with the perfluorocarbon liquid and the amphiphilic peptides of each peptide-based nanoparticle are crosslinked to each other, the resulting nanoparticles comprise a 1D fibrils morphology.

In some embodiments, any nanoparticle described herein, including nanoparticles with different morphologies, can be incorporated into a cell. For example, in some embodiments, macrophages can internalize nanoparticles when brought in contact with them in vitro. The resulting composition is then a plurality of cells, each with one or more nanoparticles contained within. Thus, in some embodiments, compositions are provided that comprise a plurality of cells, wherein each cell comprises at least one nanoparticle described herein. In some embodiments, the plurality of cells is a plurality of macrophages. Additionally, in some embodiments, a method of preparing a cellular composition comprising a plurality of cells is provided, the method comprising contacting a perfluorocarbon liquid with a plurality of amphiphilic peptides to form a plurality of peptide-based nanoparticles, wherein each peptide-based nanoparticle comprises a perfluorocarbon liquid core and a plurality of amphiphilic peptides surrounding the perfluorocarbon liquid core, and contacting the plurality of peptide-based nanoparticles with the plurality of cells, where in each cell of the plurality of cells internalizes at least one peptide-based nanoparticle.

In some embodiments, the cellular compositions comprising the nanoparticles can be used in cellular tracking by tracking the cells with nanoparticles via ultrasound. Because the nanoparticles are internalized within the cells of the composition, as those cells move throughout a tissue, their location can be tracked with the use of an ultrasound device as described herein. Thus, in some embodiments, a method of cellular tracking is provided, the method comprising administrating a cellular composition with nanoparticles described herein to a tissue, administrating ultrasonic waves to the tissue, and detecting the location of the cells in the tissue by locating acoustic properties of the peptide-based nanoparticles in the plurality of cells.

The present disclosure is illustrated and further described in more detail with reference to the following non-limiting enumerated embodiments and examples.

### ENUMERATED EMBODIMENTS

1. A composition comprising a plurality of cells,
wherein each cell comprises at least one peptide-based nanoparticle,
wherein the at least one peptide-based nanoparticle comprises a perfluorocarbon liquid core and a plurality of amphiphilic peptides surrounding the perfluorocarbon liquid core,
wherein each amphiphilic peptide is represented by Formula (III):

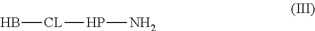

wherein HB is a fluorinated hydrophobic block consisting of three to five consecutively connected pentafluorinated hydrophobic amino acid residues;
wherein CL is an amino acid sequence consisting of two to 10 amino acid residues, at least two of which are cross-linking cysteine residues;
wherein HP is a hydrophilic amino acid sequence,
wherein said amphiphilic peptide consists of 8 to 30 total amino acid residues,
wherein the amphiphilic peptides are oriented such that groups HB of the amphiphilic peptides are interpolated into the perfluorocarbon liquid of the perfluorocarbon liquid core and groups HP extend away from the perfluorocarbon liquid core.
   2. The composition of embodiment 1, wherein each cell of the plurality of cells is a macrophage.
   3. The composition of embodiment 1 or 2, wherein the amphiphilic peptides of each peptide-based nanoparticle are crosslinked to each other through the cross-linking motif CL.
   4. The composition of embodiment 3, wherein each peptide-based nanoparticle comprises a crosslinked unimolecular monolayer morphology or a 2D sheet morphology.
   5. The composition of embodiment 1 or 2, wherein the amphiphilic peptides of each peptide-based nanoparticle are not crosslinked to each other.
   6. The composition of embodiment 5, wherein each peptide-based nanoparticle comprises a non-crosslinked unimolecular monolayer morphology or a 1D fibrils morphology.
   7. The composition of any one of embodiments 1 to 6, wherein the composition further comprises a pharmaceutically acceptable excipient selected from the group consisting of a vehicle, an adjuvant, a carrier, and a diluent.
   8. The composition of any one of embodiments 1 to 7, wherein HB consists of three, four or five consecutively connected pentafluoro-phenylalanine residues, and is located at the N-terminal end of the peptide sequence.
   9. The composition of any one of embodiments 1 to 7, wherein HP comprises lysine, glycine, arginine, aspartic acid, or any combination thereof.
   10. The composition of any one of embodiments 1 to 9, wherein HP comprises the sequence KGRGD (SEQ ID NO: 35), where K is lysine, G is glycine, R is arginine, and D is aspartic acid.
   11. The composition of any one of embodiments 1 to 10, wherein CL comprises GGGCCGG (SEQ ID NO: 46), where G is glycine and C is cysteine.
   12. The composition of any one of embodiments 1 to 11, wherein said hydrophilic amino acid sequence of HP comprises a targeting motif.
   13. The composition of any one of embodiments 1 to 12, wherein said hydrophilic amino acid sequence comprises a conserved targeting motif selected from the group consisting of: HGK, RGD, KAR, RSR, KAA, RGRR(SEQ ID NO:1), RGRRS (SEQ ID NO:2), YQLDV (SEQ ID NO:3), EYQ, RPM, PSP, VGVA (SEQ ID NO:4), NGR, CRKRLDRNC (SEQ ID NO:43), EFEEFEIDEEEK (SEQ ID NO:44), and DFEEIPEEYLQ (SEQ ID NO:45).
   14. The composition of any one of embodiments 1 to 13, wherein said hydrophilic amino acid sequence comprises a hydrophilic amino acid sequence selected from the group consisting of: KGRGD (SEQ ID NO:35), RGDS (SEQ ID NO:36), GRGD (SEQ ID NO:37), GRGDS (SEQ ID NO:38), GRGDSP (SEQ ID NO:39), GRGDSPK (SEQ ID NO:40), GRGDNP (SEQ ID NO:41), and GRGDTP (SEQ ID NO:42).
   15. The composition of any one of embodiments 1 to 14, wherein said amphiphilic peptide comprises an amphiphilic peptide represented by Formula (IV) or Formula (V): F_{F}F_{F}F_{F}GGGCCGGKGRGD (IV) (SEQ ID NO:47), F_{F}F_{F}F_{F}F_{F}GGGCCGGKGRGD-NH2 (V) (SEQ ID NO:49), wherein F_{F} is pentafluoro-phenylalanine, G is glycine, C is cysteine, K is lysine, G is glycine, R is arginine, and D is aspartic acid.
   16. The composition of any one of embodiments 1 to 15, wherein said amino acid sequence of CL consists of two to 10 amino acid residues and said hydrophilic amino acid sequence of HP consists of 3 to 15 hydrophilic amino acids, and wherein said amphiphilic peptide consists of 10 to 30 total amino acid residues.
   17. The composition of any one of embodiments 1 to 16, wherein the amphiphilic peptide has a molecular weight in a range of about 2000-5000 daltons, wherein the amphiphilic peptide comprises at least eight amino acid residues, and a total number of no more than 30 amino acid residues, wherein at least two of the amino acid residues are connected consecutively by peptide bonds without any intervening amino acid residues.
   18. A composition comprising a plurality of macrophages,
wherein each macrophage comprises at least one peptide-based nanoparticle,
wherein the at least one peptide-based nanoparticle comprises a perfluorocarbon liquid core and a plurality of amphiphilic peptides surrounding the perfluorocarbon liquid core,
wherein each amphiphilic peptide comprises F_{F}F_{F}F_{F}F_{F}GGGCCGGKGRGD-NH2 (SEQ ID NO: 49), wherein F_{F} is pentafluoro-phenylalanine, G is glycine, C is cysteine, K is lysine, G is glycine, R is arginine, and D is aspartic acid, and
wherein amphiphilic peptides are oriented such that the pentafluoro-phenylalanine region is interpolated into the perfluorocarbon liquid core and the KGRGD (SEQ ID NO:35) region extends away from the perfluorocarbon liquid.
   19. The composition of embodiment 18, wherein the amphiphilic peptides of each peptide-based nanoparticle are crosslinked to each other through a cross-linking motif GGGCCGG (SEQ ID NO: 46), and wherein each peptide-based nanoparticle comprises a crosslinked unimolecular monolayer morphology or a 2D sheet morphology.
   20. The composition of embodiment 18, wherein the amphiphilic peptides of each peptide-based nanoparticle are not crosslinked to each other, and wherein each peptide-based nanoparticle comprises a non-crosslinked unimolecular monolayer morphology or a 1D fibrils morphology.
   21. A method of preparing the cellular composition comprising a plurality of cells of any one of embodiments 1 to 20, the method comprising:
contacting a perfluorocarbon liquid with a plurality of amphiphilic peptides to form a plurality of peptide-based nanoparticles, wherein each peptide-based nanoparticle comprises a perfluorocarbon liquid core and a plurality of amphiphilic peptides surrounding the perfluorocarbon liquid core, and
contacting the plurality of peptide-based nanoparticles with the plurality of cells, wherein each cell of the plurality of cells internalizes at least one peptide-based nanoparticle.
   22. The method of embodiment 21, wherein water is added to plurality of peptide-based nanoparticles after their formation.
   23. The method of embodiment 22, wherein the amphiphilic peptides of each peptide-based nanoparticle are crosslinked to each other, forming a peptide-based nanoparticle comprising a crosslinked unimolecular monolayer morphology.
   24. The method of embodiment 22, wherein the amphiphilic peptides of each peptide-based nanoparticle are not crosslinked to each other, forming a peptide-based nanoparticle comprising a non-crosslinked unimolecular monolayer morphology.
   25. The method of embodiment 21, wherein water is added to the plurality of amphiphilic peptides prior to their contact with the perfluorocarbon liquid.
   26. The method of embodiment 25, wherein the amphiphilic peptides of each peptide-based nanoparticle are crosslinked to each other, forming a peptide-based nanoparticle comprising a 2D sheet morphology.
   27. The method of embodiment 25, wherein the amphiphilic peptides of each peptide-based nanoparticle are not crosslinked to each other, forming a peptide-based nanoparticle comprising a 1D fibrils morphology.
   28. A method of cellular tracking, comprising:
administering a composition of any one of embodiments 1 to 20 to a tissue,
administering ultrasonic waves to the tissue, and
detecting the location of the cells in the tissue by locating acoustic properties of the peptide-based nanoparticles in the plurality of cells.
   29. The method of embodiment 28, wherein the ultrasonic waves are administered to the tissue by a B-mode ultrasonic imaging device or a Doppler ultrasonic imaging device.
   30. The method of embodiment 28 or 29, wherein the ultrasonic waves induce a liquid-to-gas phase transition in the peptide-based nanoparticles that generates echogenic microbubbles inside the plurality of cells.

### EXAMPLES

### Example 1: Nanoparticle Synthesis and Characterization Methods

### Materials and General Methods

Fmoc-protected amino acids were purchased from Novabiochem. PL-Rink resin was purchased from Polymer Laboratories. 1H-Benzotriazolium 1-[bis(dimethylamino) methylene]-5chloro-hexafiuorophosphate (1-),3-oxide (HCTU) was obtained from Peptides international. Trifluoroacetic acid was obtained from Acros organics, and 1,2-ethanedithiol was purchased from Fluka. Oregon Green 514 Phalloidin, 6 and 24 well cell culture plates, polystyrene microcuvettes, diethyl ether, dimethyiformamide (DMF), acetonitrile (ACN), N-methylpyrrolidone (NMP), Slide-A-Lyzer™ dialysis cassettes (MWCO 3.5K) and 96-well half area high content imaging glass bottom micropiates were purchased from Fisher Scientific. Perfluorohexane (PFH) and perfluoropentane (PFP) were purchased from Oakwood Chemicals and Strem Chemicals, respectively. N,N-diisoproylcarbodiimide (DIC) was purchased from Chem Impex. Thioanisole, anisole, 3-(4,5-dimethyl-2-thiazolyl)-2,5-diphenyl-2H-tetrazolium bromide (MTT), 2,2′-dithiodipyridine (DTP), dimethyl sulfoxide (DMSO), diazabicyclo[5.4.0]undec-7-ene (DBU) and 200 mM glutamine solution were obtained from Sigma-Aldrich. RPMI-1640 media, Hanks Balanced Salt Solution (HISS) and Hoechst 33342 trihydrochloride dye was purchased from Invitrogen. Phosphate buffered saline (PBS) IX without calcium and magnesium, and L-glutamine (L-Gln), were purchased from Corning. Heat inactivated fetal bovine serum (FBS) and trypsin EDTA were obtained from Hyclone Laboratory Inc. HPLC solvents consisted of solvent A (0.10% TFA in water) and solvent B (0.1% TFA in ACN) Gentamycin was purchased from VWR. RPMI-1640 without L-glutamine was purchased from Lonza. Hoechst 33342 and UltraPure™ agarose were purchased from Invitrogen. 4% paraformaldehyde in PBS was purchased. from Chem Cruz. The green fluorescent protein (GFP, 36 kDa) was obtained from Dr. J. P. Schneider (Chemical Biology Laboratory, NCI) and A549 human cancer cell line was obtained from the NCI-60 repository. All peptides utilized for experiments were prepared with an amidated C-terminus.

### Peptide Synthesis

Fmoc-based solid-phase peptide chemistry was used to prepare the amphiphilic peptides, with HCTU activation on PL-Rink resin using an automated ABI 433A peptide synthesizer. Amphiphilic peptides were cleaved from the resin and simultaneously side-chain deprotected using a trifluoroacetic acid/thioanisole/1,2-ethanedithiol/anisole (90:5:3:2) cocktail for 2 hours under argon atmosphere. The crude product was precipitated with cold diethyl ether and then lyophilized. Amphiphilic peptides were purified via reverse-phase HPLC equipped with a FluoroFlash® semi-preparative column composed of silica gel bonded with perfluorooctylethylsilyl (Si(CH2)2C8F17). A gradient of 0-50% solvent B over 25 min., followed by 50-100% solvent B over an additional 50 min. was utilized. All amphiphilic peptides were lyophilized to collect the pure product, and the purity verified by analytical HPLC-MS.

### Nanoparticle Formation

Peptides were weighed out as a dry fluffy solid in a round bottom flask, and dissolved in a volume of 1:1 DMF:ACN containing 1% TFA to a final concentration of 0.5-2.0 mg/mL. The solution was stirred at 1,000 rpm on ice for 15 min. before addition of 1%-2% (v/v) cold PFP. After an additional 5 min. of stirring to properly mix the components and create an emulsion, an equal volume of cold MilliQ water was slowly added dropwise. During this solvent exchange procedure the solution turned opaque due to self-assembly of the peptides at the interface of the water-PFP emulsion. The mixture was stirred at 1,000 rpm for 1 hour on ice, over which time the solution clarified. Unincorporated peptide was removed by dialyzing the mixture against MilliQ water containing 2.5% (v/v) DMSO to oxidize the cysteines and facilitate disulfide cross-linking of the amphiphilic peptides of the nanoparticles. In addition, a Pasteur pipette was used to gently bubble air into the media to further promote oxidation. Dialysis was performed for 12 hours, with exchanges every four hours. Two final exchanges of the dialysis media to pure MilliQ water, for 2 hours each, removed residual DMSO. The purified nanoparticles were removed from the dialysis cassette, placed into a clean glass vial and used for experiments within 48 hours.

### Physico-Chemical Characterization

Particle size and zeta potential measurements were performed via dynamic light scattering using a Zetasizer Nano-ZS instrument (Malvern, Worcestershire, UK). For size determination, a solution of nanoparticle sin water was diluted times into characterization buffer (25 mM Tris-HCl, 150 mM NaCl, pH 7.4) to reach a final volume of 1 mL in a clean polystyrene microcuvette. The size of pure 1-2 vol % PFP emulsions prepared in 1:1 DMF:ACN containing 1% TFA were also measured as controls. Three independent measurements, ten runs each, were taken at a 175° scattering angle, a sample position of 4.65 mm and an attenuation of 11. Particle size was recorded at both 25° C. and 37° C., with a 2 min. sample equilibration time. Material refractive index (RI) was set at 1.59 (25° C.) and 1.45 (37° C.) using pre-defined settings provided by the manufacturer. Dispersant RI of 1.332 and viscosity [cP] equal to 0.9103 (25° C.) and 0.7096 (37° C.) were calculated using the ‘Solvent Builder’ tool in the Zetasizer software. Phase analysis light scattering (PALS) assisted zeta potential measurements were performed by adding the solution of nanoparticles to MilliQ water to achieve a ten-fold dilution and loading 700 μL of the sample into a disposable folded capillary cell (Malvern, DTS1070). Three independent measurements were taken at 25° C., with twenty runs each.

In separate studies, the stability of nanoparticles during storage was evaluated via dynamic light scattering. Here, purified particles (formulation B of Table 1) were dispersed into milliQ water and left at room temperature. At defined time points over 15 days an aliquot was removed, diluted ten times into characterization buffer, and particle size and count rate recorded at 25° C. Of note, count rate was used as a qualitative indicator of particle density and thus an estimate of stability over time. In parallel experiments, the same particles were initially diluted ten times into blank characterization buffer, or buffer supplemented with 5% fetal bovine serum, and incubated at 37° C. to evaluate their stability under physiologic conditions. At defined time points over 48 hours a 1 mL aliquot was directly added to a clean polystyrene microcuvette and particle size measured at 37° C. For both experiments, three independent measurements were taken with twenty runs each.

### Nanoparticle Visualization

Differential interference contrast (DIC) microscopy was used to image the nanoparticles in solution. Briefly, nanoparticles were diluted two times into characterization buffer and added to 96-well glass bottom high-content imaging microplates. The plates were then loaded onto an LSM 710 confocal microscope (Zeiss, Thornwood, NY) equipped with a temperature controlled humidified chamber. Images were collected at 25° C. and 37° C., with a 15 min. sample equilibration time, using a 63× Plan-Apochromat oil objective.

### Disulfide Formation Assay

A 1.5 mL solution of freshly prepared nanoparticles in water (0.5 mg/mL peptide and 2% PFP) was placed in a round bottom flask and slowly stirred with gentle bubbling of air. A 2.5% volume of DMSO was added to oxidize the thiols and initiate cross-linking. At specific time points, a 30 μL aliquot of the mixture was diluted into 200 μL of 0.1 mM DTP in characterization buffer, and allowed to react for 10 min. The solution was then transferred to a quartz cuvette (1 cm pathlength) and concentration of the free thiolate was determined via absorption at 343 nm (E343=7600 cm⁻¹ M⁻¹) (Haines et al., 2005) using an Agilent 8453 UV-Vis spectrophotometer (Santa Clara, CA). In separate control experiments, the same procedure was followed without the addition of 2.5% (v/v) of DMSO to evaluate its influence on thiol oxidation and disulfide cross-linking of nanoparticles. All values were corrected for background DTP hydrolysis. Percentage of disulfide formation was calculated by subtracting the concentration of free thiolate from the initial cysteine concentration. Studies were performed in triplicate.

### Cell Viability Assay

A549 cells were seeded onto 6 well plates at 5×10⁵ cells/well and allowed to adhere overnight. Cells were then washed and 3 mL of warm HBSS added to each well. Ultrasound was applied for 90 see at a duty cycle of 10%-20% with intensity varied between 0.1-1.0 W/cm², corresponding to a peak negative pressure of 0.054-0.172 mPa, respectively. Wells not subjected to US, or cells incubated with 25% DMSO in HBSS for 1 hour, were used as negative and positive controls, respectively. Following US insonation, cells were incubated for 30 min. to recover and then washed with warm HBSS. 3 mL of a 0.5 mg/mL solution of MTT reagent in HBSS was added to each well and incubated for 2 hours. The supernatant was removed and replaced with 3 mL of DMSO to dissolve the formazan product, followed by transfer of a 100 μL aliquot of the colored solution to a 96 well plate. Absorbance was then read at 540 nm using a UV plate reader (Biotek, Winooski, VT). The absorbance of negative controls was subtracted from each sample as a blank, and percent viability calculated using the equation: (Absorbance US-treated cells/Absorbance untreated cells)×100. Results shown represent the average of three independent experiments standard deviation.

### Results

As evidenced by the above data, the de novo designed peptide F_{F}F_{F}F_{F}GGGCCGGKGRGD-NH₂ (SEQ ID NO:49) is capable of assembling at the surface of a perfluoro-n-pentane (PFP) droplet. The peptide sequence contains three pentafluoro-phenylalanine (F_{F}) residues at its N-terminus, which promotes interpolation and assembly of the peptide at the PFP-liquid interface. C-terminal to this fluorous domain is a cysteine containing motif, GGGCCGG (SEQ ID NO:46), designed to undergo disulfide cross-linking to stabilize the peptide corona after templated assembly. Incorporation of a bioactive hydrophilic sequence at the peptide's C-terminus ultimately leads to its multivalent display at the surface of the assembled particle. In this particular design, the sequence KGRGD (SEQ ID NO:35) has been included to enable cell-surface localization of the nanoparticle mediated by binding of RGD with extracellular integrins. Despite inclusion of highly fluorinated residues, this sequence was able to be chemically synthesized in high yield and purity using standard solid-phase techniques.

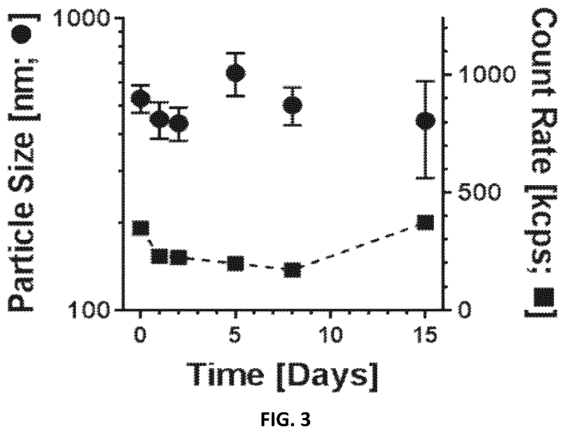

It has been found that, to form nanoparticles, a solvent-exchange procedure in which cold water is slowly added to an organic emulsion of amphiphilic peptides and PFP, ultimately leads to spontaneous assembly of the amphiphilic peptides at the surface of PFP liquid core. Importantly, this mild procedure eliminates the need for aggressive synthetic methods commonly used to prepare stimuli-responsive particles, which can lead to degradation of the encapsulated cargo. Subsequent dialysis against 2.5% DMSO in water removes unincorporated peptide, and promotes disulfide cross-linking of cysteine residues in the perfluorocarbon liquid core corona. Cross-linked nanoparticles remain stable for multiple weeks when stored at room temperature in water (FIG. 3).

It has also been found that the size of nanoparticles could be precisely controlled between 250 nm and 1,200 nm, as a function of peptide and PFP feed ratio (FIG. 6, Table 1).

TABLE 1
Physicochemical properties of nanoparticle formulations

| | Amphiphilic | PFP | | | | |
|---|---|---|---|---|---|---|
| | Peptide Conc. | [vol | Droplet size [nm] | | | ζ _{[c]} |
| Formulation | [mg/mL] | go] | 25° C. | 37° C. | Δ _{[b]} | [mV] |
| E | 1.00 | 2 | 1.2 × | 43 × | 36.5 | 3.5 ± |
| | | | 10³ | 10³ | | 0.2 |
| D | 1.00 | 1 | 863 ± | 23 × | 26.7 | 0.1 ± |
| | | | 98 | 10³ | | 0.2 |
| C | 0.75 | 2 | 739 ± | 7.5 × | 10.1 | 6.8 ± |
| | | | 56 | 10³ | | 0.5 |
| B*^{[a]} | 0.75 | 1 | 469 ± | 482 ± | 1.0 | 8.0 ± |
| | | | 24 | 34 | | 0.4 |
| B | 0.50 | 2 | 453 ± | 528 ± | 1.3 | 8.2 ± |
| | | | 29 | 24 | | 0.4 |
| A*^{[a]} | 0.25 | 2 | 448 ± | 458 ± | 1.0 | 4.6 ± |
| | | | 27 | 23 | | 0.2 |
| A | 0.50 | 1 | 301 ± | 390 ± | 1.3 | 12.4 ± |
| | | | 14 | 12 | | 0.4 |

^{[a]}Formulations A* and B* are not shown in FIG. 6 due to their similar size to formulation B.
_{[b]} fold change in particle size at 37° C. versus 25 oC.
_{[c]} zeta potential.

Dynamic light scattering performed on pure PFP emulsions indicates this may be due, in part, to different sizes of PFP droplets formed in the starting emulsion (FIG. 4). At any rate, the ability to control the hydrodynamic radii of the particles is critically important for delivery applications, as this parameter is inversely correlated with passive tissue distribution, and directly proportional to the US magnitude required for droplet cavitation.

Also evaluated was the influence of temperature on nanoparticle size through direct visualization of particles in solution using differential interference contrast (DIC) confocal microscopy, as well as dynamic light scattering analysis. Results show that nanoparticles with a diameter<750 nm at 258° C. were able to maintain their size when heated to physiologic temperature, a vital requirement for acoustic droplet vaporization in vitro and in vivo (Shpak et al., 2014). Exceeding this size threshold led to premature PFP vaporization (bp=29 8° C.) and converted the perfluorocarbon liquid cores into gaseous microbubbles at 37° C., as evident by the massive increase in diameter for the purple, green and orange formulations. This influence of particle size on the vaporization temperature of PFP is due to the inverse relationship between internal pressure and droplet dimension, as described by the Laplace pressure Equation (1):

$${P}_{in} = \frac{2 σ}{{R}_{H}} + {P}_{atm} \qquad ( 1 )$$

where Pᵢₙ and Pₐₜₘ are the internal droplet pressure and atmospheric pressure, respectively, is the interfacial surface tension and RH represents the hydrodynamic droplet radii. Here, decreasing the droplet size leads to an increase in the pressure exerted on the PFP core, ultimately keeping the fluorous liquid in a superheated state well above its bulk boiling point of 29° C. The influence of vapor pressure on the temperature of the PFP solvent can be defined using the Antoine vapor Equation (2):

$$T = \frac{B}{A - {log}_{10} P} - C \qquad ( 2 )$$

in which T and P represent temperature and pressure, respectively, while A, B, and C are equation parameters empirically determined for PFP (Barber et al., 1956). Combining the Laplace pressure (1) and Antoine vapor (2) equations provides a single expression describing the temperature at which the vapor pressure of the core is equal to the internal droplet pressure (Tᵥₐₚ), ultimately causing thermal droplet vaporization (3):

$${T}_{vap} = \frac{B}{A - {log}_{10} ( \frac{2 σ}{{R}_{H}} + {P}_{atm} )} - C \qquad ( 3 )$$

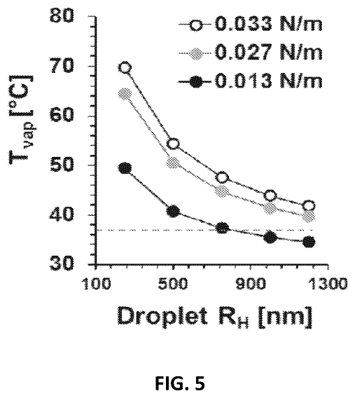

Using this equation, the relationship between Tvap and droplet size can be modeled using reported surface tension values for PFP emulsions formulated with either BSA (0.033 Nm⁻¹), the amphiphilic polymer PEO-PLA (0.027 Nm⁻¹), or the cationic surfactant cetrimonium bromide (CTAB; 0.013 Nm⁻¹) (FIG. 5) (Kandadai et al., 2010). Of note, the PFP-CTAB formulation most closely resembles the nanoparticles reported here, in which the cationic amphiphilic sequence acts as the surfactant. Results from the model show that, at a surface tension of 0.013 Nm⁻¹, the vaporization point of the PFP core is expected to be >378° C. when particles are <800 nm in size, a finding that closely matches the experimental threshold identified for the PFP-peptide emulsions.

This suggests that the US energy required to thermally vaporize the nanoparticle core could be carefully controlled by modulating the droplet size, as well as changing the interfacial surface tension through tuning the amphiphilic character of the assembling peptide.

As described herein, a new class of peptide-based nanodroplets, nanoparticles, capable of ultrasound-mediated delivery of membrane-impermeable cargo into cells has been developed. In this example, nanoparticles are prepared via the de novo designed peptide F_{F}F_{F}F_{F}GGGCCGGKGRGD-NH₂ (SEQ ID NO:49), which efficiently assembles at the surface of organofluorine droplets, and undergoes cysteine-mediated cross-linking to stabilize the final nanostructure. Biomolecular cargo can be readily encapsulated within the nanoparticle carrier during the assembly process. Cell binding of the nanoparticles, followed by acoustic vaporization, ultimately delivers the cargo into cells. Gaseous microbubbles generated during vaporization of nanoparticles may also function as an US contrast agent to allow for imaging and guidance of the delivery modality in real-time. Thus, nanoparticles of the present invention represent a potential theranostic system with broad applications in drug delivery and biomedical imaging.

### Example 2: Visualizing NP_{gfp} Tracking and Vaporization by Ultrasound Imaging

Ultrasound waves allow for image-guidance and temporal vaporization of NP_{gfp} for protein delivery.

For ultrasound B-mode and Doppler imaging, NP_{gfp} prepared as above were diluted 1:10 in degassed DI water. A 1.5% agarose phantom was degassed before gelation into mold of 50 ml beaker with 1 cm glass tube imprint (˜2 cm deep within gel). The agarose phantom was placed on a block of neoprene, an acoustic absorbing material, in a large bucket of degassed water. NP_{gfp} were imaged in B-mode and Doppler using L7-4 (5 MHz, 3 cycles) and L22-14v (18.5 MHz, 12 cycles) transducers. Using the Verasonics Matlab script, the voltage of the transducers was incrementally increased for image-guided particle tracking in B-mode and for particle acoustic droplet vaporization with Doppler mode. Video images were captured and In-phase quadrature (I/Q) Doppler data was saved throughout the entire experiment. The L7-4, 5 MHz transducer, pressures were measured using a hydrophone. The pressure threshold of acoustic droplet vaporization was performed for three replicates for average ultrasound pressure necessary for droplet vaporization.

Real-time monitoring of NP_{gfp} was captured with B-mode imaging from an 18.5 MHz transducer. Three individual NP_{gfp} were tracked with high echogenicity over six seconds at one second time intervals. This demonstrates that NP_{gfp} are capable of use as ultrasound image-guided particles.

Additionally, the temporal vaporization of NP_{gfp} was captured with Doppler mode imaging from a 5 MHz transducer.

### Example 3: In Situ Imaging of Macrophages Via Phase-Change Peptide Nanoemulsions

### Background

Macrophages are phagocytic cells that play key roles in innate and adaptive immunity. They are important cellular mediators of wound healing and tissue regeneration and contribute to inflammatory-linked diseases that include diabetes, atherosclerosis, and rheumatoid arthritis. These varied effector functions have spurred interest in developing modified and engineered macrophage cell therapies, particularly for oncology.

However, like all cell-based therapies, variable biodistribution profiles and tissue migration patterns can lead to heterogenous therapeutic responses and increase the risk of unwanted side effects. Accordingly, technologies that enable real-time and long-term imaging of macrophages would allow clinicians and scientists to monitor tissue distribution in situ to inform treatment approaches, predict and avoid off-target toxicities, and ultimately enhance our understanding of the dynamic behavior of these cells. Furthermore, real-time and non-invasive monitoring of these cells with spatiotemporal resolution will improve patient outcomes during treatment with adoptive or allogenic products and may reveal previously unknown activities of macrophages during disease progression.

Toward this goal, ultrasound (US) is inexpensive, portable, radiation-free, and provides real-time acquisition in a non-invasive manner with excellent penetration depth and spatial resolution when paired with microbubble acoustic contrast agents. However, traditional bubble-based agents used for ultrasonography cannot be readily coupled to macrophages due to low uptake efficiency and have short persistence time in circulation due to gas diffusion into blood.

Phase-changing nanoparticles that can be acoustically activated to form bubble-based nuclei within macrophages permit in situ imaging of these cells using traditional diagnostic US modalities. This advance is made possible by controlling the assembly pathway of the fluorinated peptide emulsifier at the droplet surface, ultimately leading to formulations that are efficiently internalized into macrophages and stably oscillate under B-mode and Doppler US imaging. A noteworthy advantage of these peptide emulsifiers, relative to traditional lipids and polymers, is that their sequences can be readily tuned to possess varying cross-linking moieties and cell-targeting motifs, without the necessity of secondary chemical modification. As a result, emulsifying peptides can be rationally designed to target a range of cell-types for application-specific requirements, while exploiting the synthetic tractability of solid-phase synthesis techniques.

The following experiments describe liquid phase-changing nanoparticles that are readily internalized into macrophages and undergo a US directed liquid-to-gas phase transition to convert into echogenic microbubbles in situ. This permits on-demand, real-time, and continuous acoustic imaging of macrophages in tissues with high spatiotemporal resolution. Modulating the interfacial assembly of the stabilizing emulsifier allows investigators to produce nanoparticles with varied surface morphologies and, as a result, tunable acoustic properties and in cellulo stability. These findings highlight the potential of this platform to open new imaging-guided approaches to improve the precision, safety, and efficacy of cell-based therapeutics.

### Development and Characterization of Peptide Emulsions

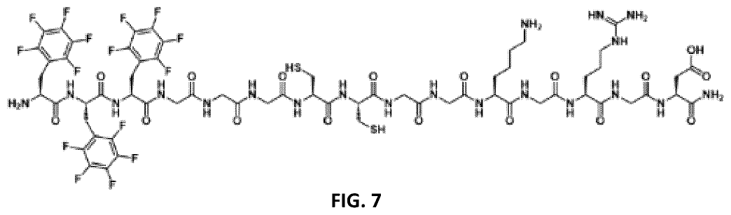

To generate the nanoparticle contrast agent, and facilitate macrophage uptake, an integrin-targeting peptide emulsifier that self-assembles at the surface of perfluorocarbon (PFC) nanodroplets: F_{F}F_{F}F_{F}GGGCCGGKGRGD-NH2 (SEQ ID NO:49) (hereafter referred to as F_{F}-RGD, F_{F}: pentafluorophenylalanine (F5-Phe), G: glycine, C: cysteine, K: lysine R: arginine, and D: aspartic acid) was utilized (FIG. 7). The fluorinated N-terminus promotes assembly of the peptide at PFC-water interfaces, while two cysteines within the central glycine-rich spacer allow for intermolecular disulfide-crosslinking between adjacent peptides to stabilize the formed particle. Finally, a C-terminal RGD motif is displayed from the surface of the emulsion to enable binding with αVβ3 integrins highly expressed on the surfaces of macrophages to promote cellular internalization.

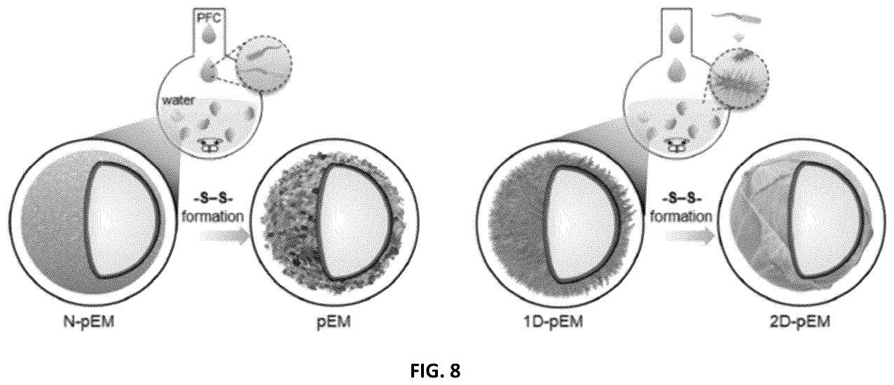

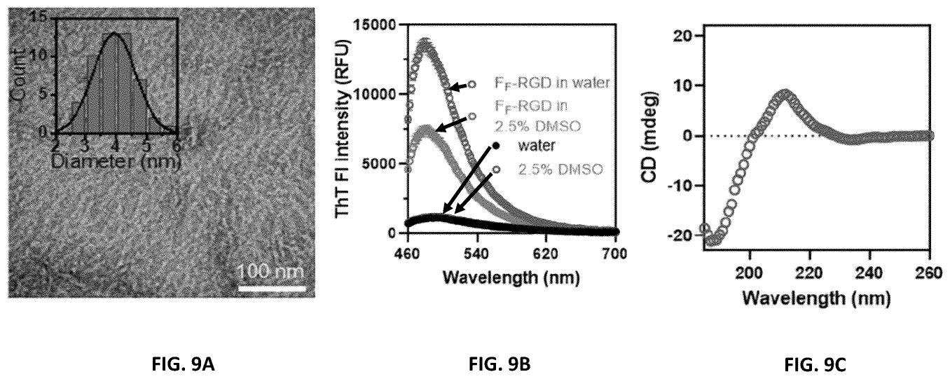

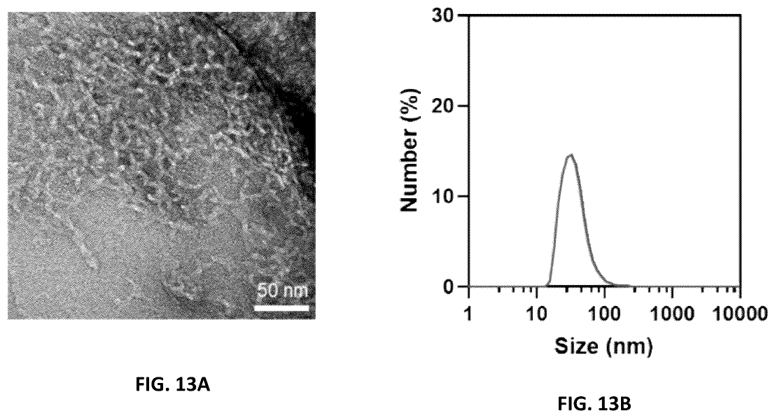

Next, varying assembly conditions were utilized to generate four distinct morphologies of the nanoparticles (FIG. 8). Using perfluorohexane (PFH) as the PFC droplet template, the FF-RGD emulsifier was dispersed within the fluorous solvent and the mixture was added to water. Spontaneous assembly of the emulsifier at the PFH droplet surface led to the formation of emulsions with a uniform surface monolayer. The resultant particles could then be left non-crosslinked (N-pEM) or further stabilized by oxidizing the cysteine residues in the presence of dimethyl sulfoxide (DMSO) to form disulfide crosslinked particles (pEM). Reversing the formulation procedure produced divergent surface morphologies. For example, dispersing F_{F}-RGD in water first, before the addition of PFH, generated 1D protofibrils≈4 nm in width and 38 nm in length (FIG. 9A; FIGS. 13A-13B). This pre-assembly is likely directed by fluorophilic interactions between F5-Phe residues to sequester the fluorinated block away from the bulk water solvent, generating fibrillar assemblies. Treating the nanofibrils with thioflavin T (ThT), a dye that displays enhanced fluorescence upon binding to amyloid structures, suggests these fibrils are β-sheet rich in nature (FIG. 9B). Yet, a canonical β-sheet signal was not observed when performing circular dichroism (CD) spectroscopy (FIG. 9C), which was expected to yield a minimum centered at ≈216 nm. Instead, a positive peak at 212 nm was observed, unusual given the L-chiral nature of this peptide and that D-chirality is typically required to produce positive CD ellipticities. Careful review of the literature suggests this CD feature can be attributed to n→π* transitions that occur due to stacking of aromatic groups within the assembling F5-Phe residues.

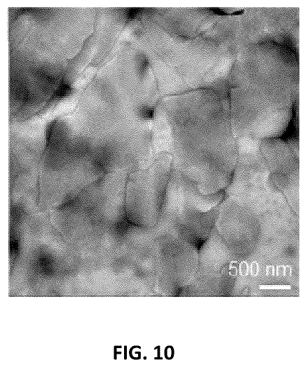

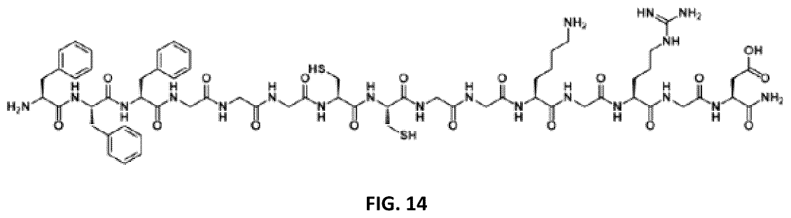

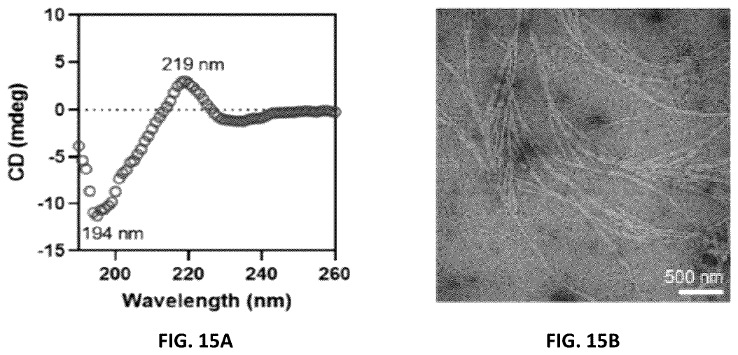

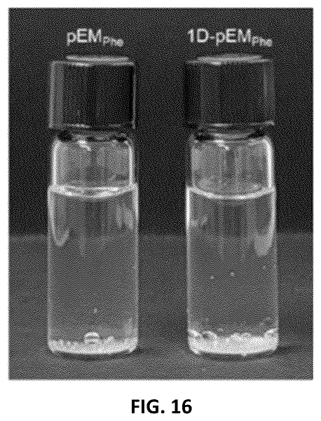

To further investigate this unusual CD profile, and specifically isolate fluorine-fluorine driven effects, an analogous sequence (F-RGD) containing natural, non-fluorinated, Phe residues was synthesized (FIG. 14). A CD spectrum similar to F_{F}-RGD was observed, with the exception that the positive peak was redshifted by 7 nm (FIG. 15A); likely due to the absence of fluorine-driven aggregation. Although CD profiles between the two peptides were similar, TEM analysis showed F-RGD assembly produced long, highly persistent fibrils, several microns in length (FIG. 15B). Interestingly, emulsification using the non-fluorinated F-RGD peptides also did not yield stable emulsions (FIG. 16), suggesting the immiscibility of natural Phe residues in the fluorous solvent prevented their interfacial assembly. With this in mind, attention was focused on F_{F}-RGD. Disulfide crosslinking of F_{F}-RGD nanofibrils in the presence of DMSO generated ordered 2D sheets was observed (FIG. 10). These sheets displayed reduced amyloid signals relative to the fibrils (FIG. 9B), possibly due to a more crowded intermolecular arrangement that inhibited intercalation of the ThT dye. Collectively, the data suggest a combination of aromatic and fluorine-fluorine interactions promotes the organization of F5-Phe into highly rigid 1D fibrillar assemblies in water, which then can be further laminated into 2D sheets via intermolecular thiol crosslinking.

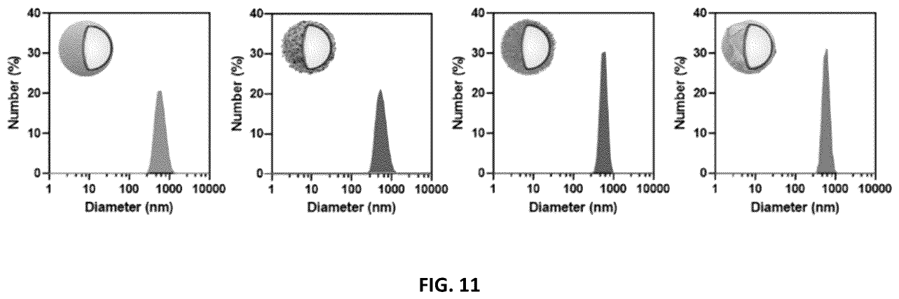

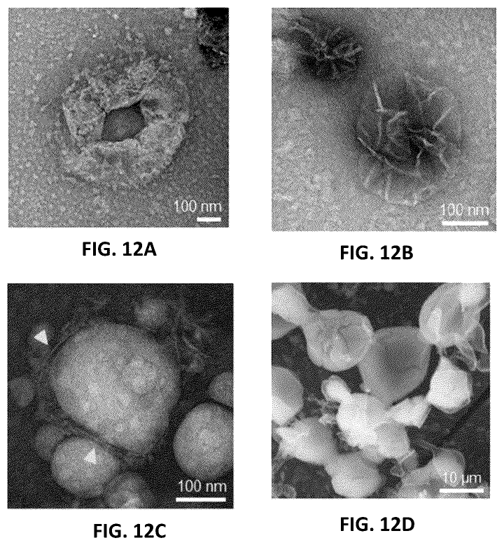

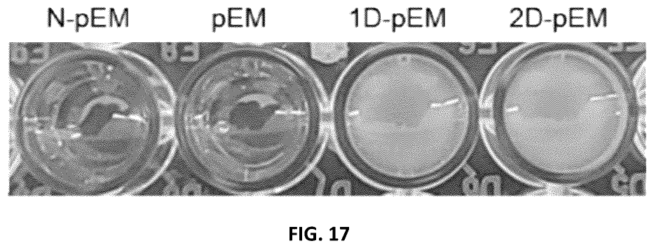

Utilizing these varied peptide assembly morphologies (e.g., non-crosslinked/crosslinked unimolecular monolayers, 1D fibrils, and 2D sheets), four distinct emulsions were developed (FIG. 8). All four formulations produced particles of ≈600 nm in diameter (FIG. 11). However, 1D-pEM and 2D-pEM yielded narrower particle size distributions and more opaque solutions, indicative of higher particle yield (FIG. 17), relative to N-pEM and pEM. This may be because 1D-pEM and 2D-pEM are formed via accumulation of solid particulates at the fluorous-water interface, a structure known as a Pickering emulsion, while N-pEM and pEM are stabilized by monomeric surfactants. Prior studies suggest that solid particles irreversibly adsorb to oil-water interfaces and generate more stable emulsions compared to monomeric surfactants (Aveyard et al., 2003), the latter being in dynamic equilibrium between bulk solvation and interfacial organization (Zhao et al., 2020). Finally, TEM and cryo-SEM imaging enabled visualization of the final emulsion structure for all four formulations (FIGS. 12A-12D).

### Acoustic Activation and Stability

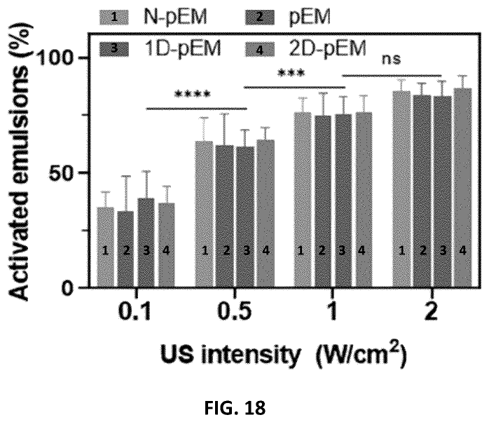

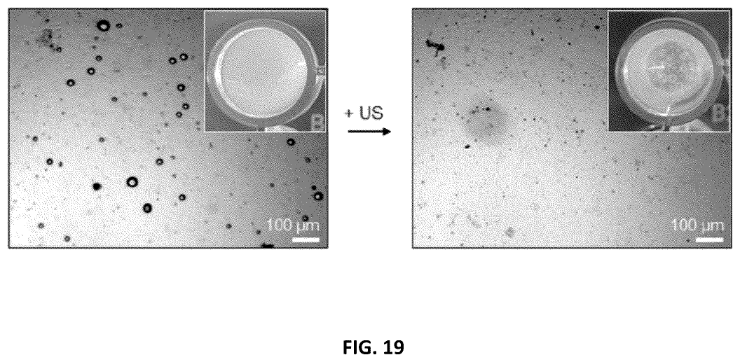

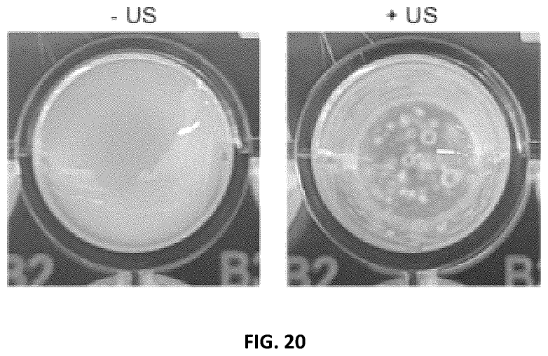

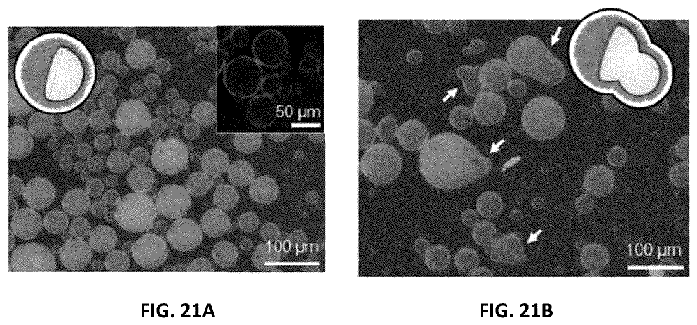

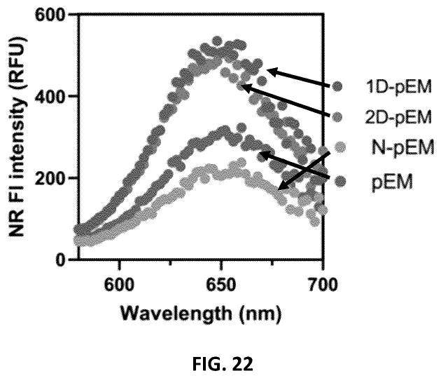

How the varied surface morphologies of the F_{F}-RGD nanoparticle phenotypes influenced their acoustic sensitivity was investigated next (FIGS. 18-20). For these studies, perfluoropentane (PFP) was selected as the fluorous core due to its decreased vaporization point (TB=29° C.) relative to PFH (TB=56° C.). This allows for lower US energies to be used to induce liquid-to-gas transitions and cause acoustic vaporization, thereby minimizing the potential for acoustic damage to tissues in future applications. Results show that all four nanoparticle formulations were readily activated at low US intensities (0.1-0.5 W cm-2), achieving ≈40-65% droplet vaporization (FIG. 18). Above 1 W cm-2, up to 85% activation was achieved. Vaporization of the nanoparticles was further confirmed by optical (FIG. 19) and fluorescence microscopy (FIGS. 21A-21B), which was limited to micron-sized nanoparticles due to magnification limitations. For fluorescence visualization, the hydrophobic dye Nile red was loaded into the liquid interior of 1D-pEM nanoparticles. Interestingly, Nile red dissolved within the PFP emulsion core had a blueshifted emission spectra, resulting in green fluorescence, while the dye localized at the surface of the nanoparticles was characteristically red (see inset of FIG. 21A). These spectral shifts are explained by the varied solvatochromic properties of Nile red based on the polarity of the bulk solution. An additional slight blue shift was observed in Nile red's emission spectra upon interaction with F_{F}-RGD fibrils assembled at the surface of 1D-pEM and 2D-pEM formulations relative to the monolayer particles, likely due to the difference in assembled structures (FIG. 22). Fortunately, these divergent spectral phenomena allowed for the independent monitoring of the dynamics of both the nanoparticle core and surface assemblies. Microscopy images shown in FIG. 21A demonstrate that, before sonication, 1D-pEM nanoparticles are intact and do not show signs of coalescence. However, after US exposure (1 W cm⁻², 90 s) the particle density is reduced, indicating vaporization (FIG. 21B). Occasional droplet coalescence was also observed, which may result from recondensation of vaporized bubbles back into fused liquid emulsions. Due to the inverse relationship between particle size and acoustic vaporization power, these larger coalesced particles would be more readily vaporized on subsequent US exposures. As will be shown later, this nanoparticle recycling is believed to play a role in the persistence of contrast-enhancing microbubble nuclei for continuous and long-term acoustic imaging.

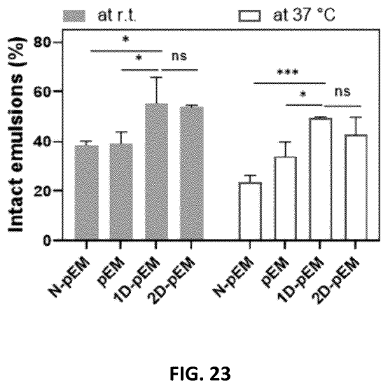

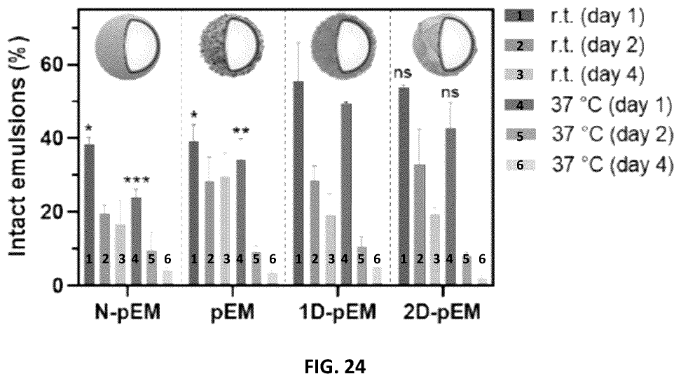

Finally, the stability of nanoparticles at room temperature and 37° C. over a 1-4 day incubation period was measured (FIGS. 23-24). Results show the Pickering nanoparticles (1D-pEM and 2D-pEM) are generally more stable than their monomeric counterparts (N-pEM and pEM). This can be explained by the irreversible adsorption of solid peptide nanostructures at the PFP-water interface, which provides a strong steric barrier against solvent diffusion and particle coalescence pre-sonication. However, unexpectedly, 1D-pEM was nominally more stable than 2D-pEM under physiologic conditions. This was surprising given that the 1D-pEM emulsion shell is not stabilized by disulfide crosslinking. As Pickering nanoparticle stability depends on the shape/morphology of the particles adsorbed at the interface, this counterintuitive result may be explained by more efficient adsorption of the short F_{F}-RGD nanofibrils to the PFP-water interface relative to the much larger 2D sheets. The varied surface morphologies may also alter the presentation of RGD cell-targeting ligands and consequently affect nanoparticle uptake into macrophages; an assertion tested in vitro.

### Macrophage Uptake and Persistence

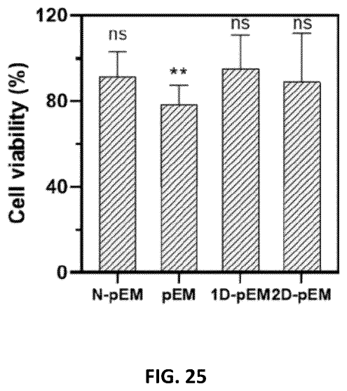

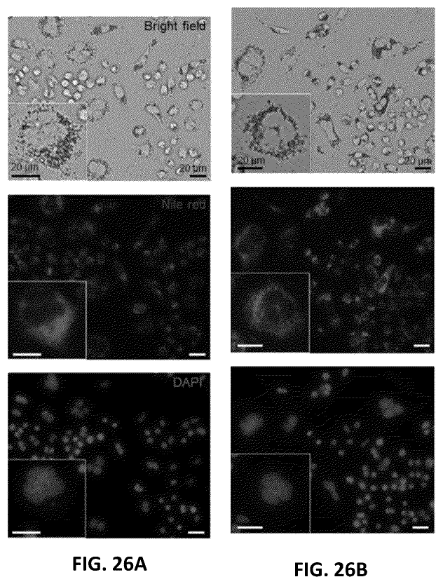

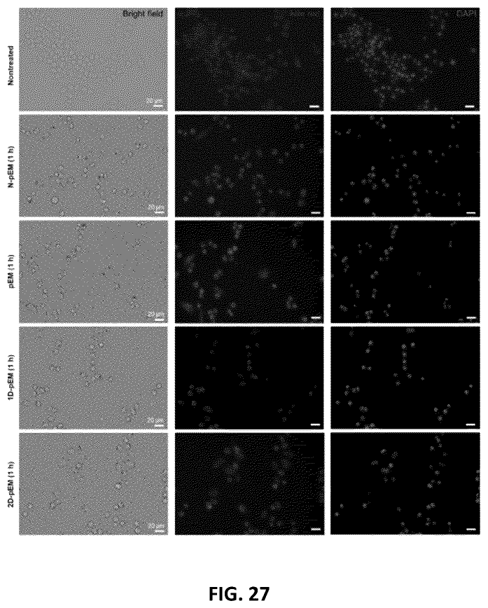

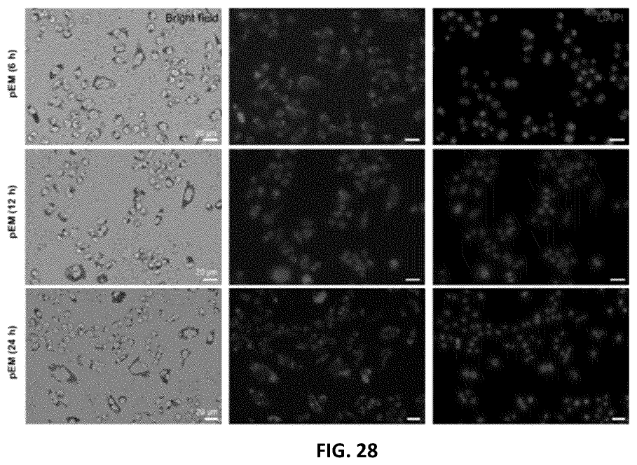

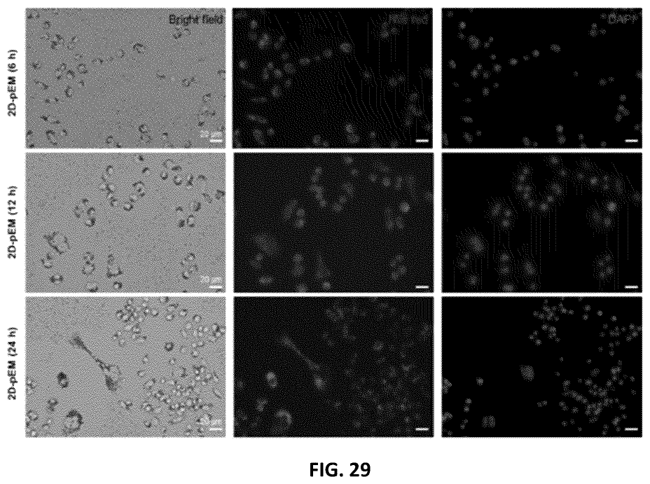

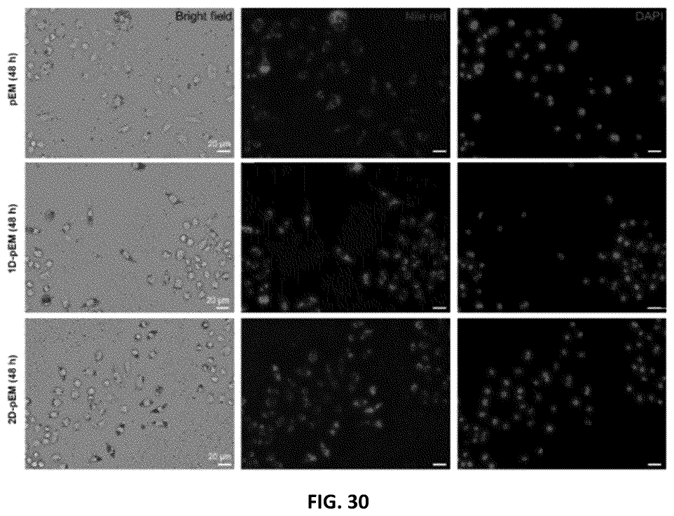

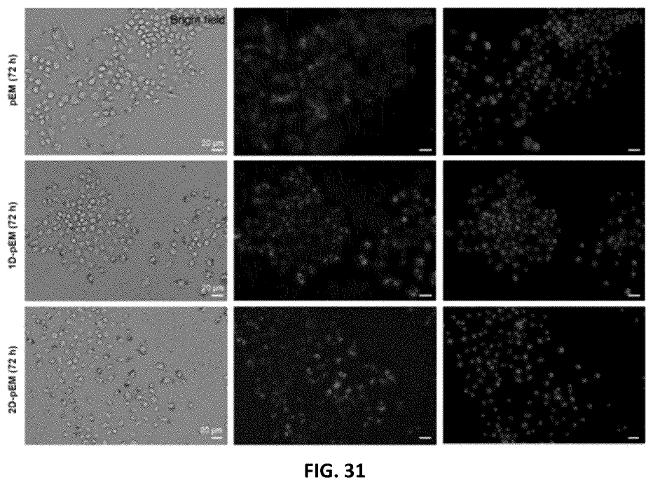

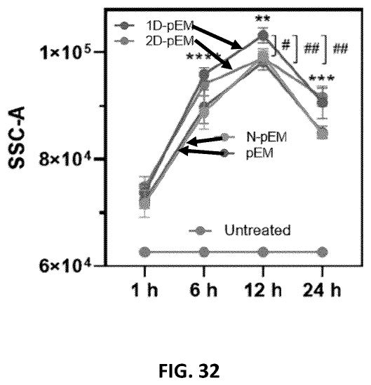

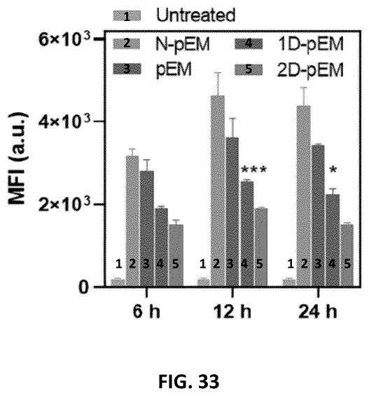

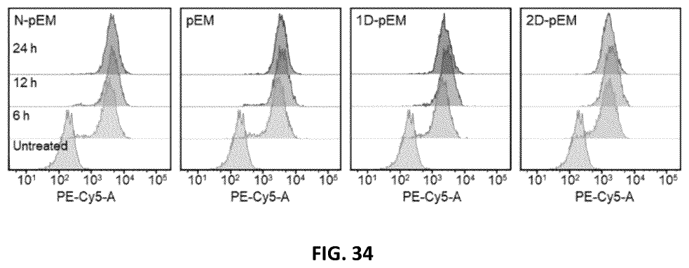

Prior to studying phagocytosis, biocompatibility of each nanoparticle formulation was assessed after a 24 h incubation with RAW 264.7 murine macrophages at the highest working concentration of the particles (FIG. 25). No significant loss of cell viability was observed. With this confirmed we next assessed the time-dependent persistence of Nile red-loaded nanoparticles in RAW 264.7 cells via epifluorescence microscopy (FIGS. 26A-26B and FIGS. 27-31). Brightfield and fluorescence micrographs taken at 1 h show that nanoparticles are rapidly internalized into macrophages (FIG. 27), and peak in intracellular load at 12 h of incubation, as confirmed by flow cytometry (FIGS. 32-34). The particles subsequently persist within the cells for >3 days (FIGS. 30-31). It is worth noting that, depending on the analysis method used, significant differences in performance between the nanoparticles are observed. For example, interpreting the side scattering data, which provides a representation of internal cellular density due to particle localization, indicates that 1D-pEM nanoparticles show superior intracellular persistence (FIG. 32). Conversely, measuring Nile red fluorescence (FIG. 33) suggests that the monomeric formulations (N-pEM and pEM) outperform the Pickering nanoparticles (1D-pEM and 2D-pEM). The side scattering data is believed to be more representative of nanoparticle performance given that the instability of the monomeric nanoparticles (FIG. 34) may lead to premature leakage of the Nile red dye, and consequently exaggerated cellular fluorescence results. The superior internalization and persistence of 1D-pEM nanoparticles may be explained by a combination of factors. First, 1D-pEM showed the greatest stability at physiological temperature compared to the other three formulations (FIG. 34; FIG. 24). Additionally, the fibrillar assembly of FF-RGD peptides may make it both resistant to proteolytic degradation and able to multivalently display RGD peptide motifs at the particle surface to maximize interactions with the αvβ3 receptor on the macrophages.

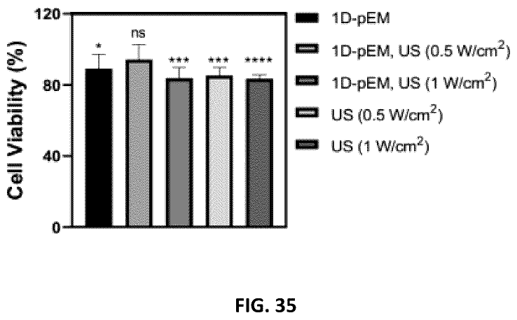

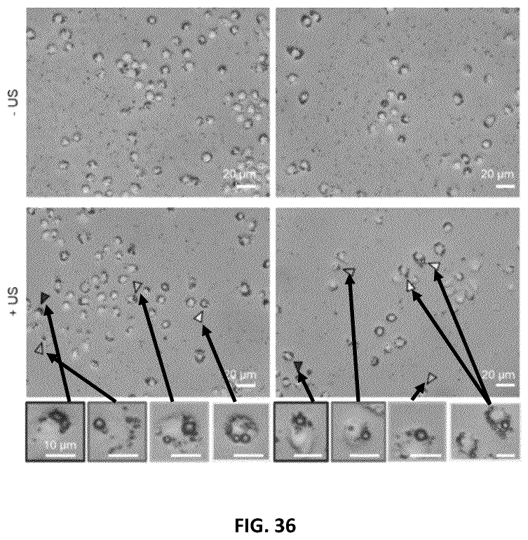

Based on the dense intracellular accumulation of particles, whether US-induced emulsion vaporization would compromise macrophage cell viability was evaluated next. To study this, RAW 264.7 cells were incubated with 1D-pEM for 12 h, followed by treatment with 1 MHz US at different intensities. We observed no significant change in viability for cells with internal vaporization of 1D-pEM compared to controls exposed to US alone (FIG. 35). The ability of these cells to tolerate intracellular generation of cavitating bubbles may be due to the combination of high cytoplasmic viscosity and local crowding by intracellular organelles and other emulsions. Together, this may constrain the size of vaporized microbubbles and thereby create gaseous nuclei that are sufficiently small to prevent intracellular damage, as further evidenced by optical imaging of 1D-pEM treated RAW 264.7 cells after US insonation (FIG. 36).

### In Vitro and Ex Vivo US Imaging of Emulsion-Laden Macrophages

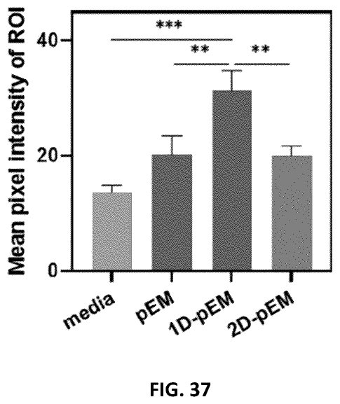

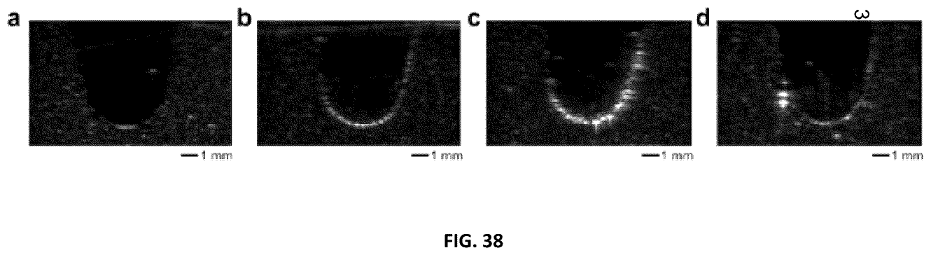

Evaluating the imaging performance of the nanoparticles was first conducted using high-resolution B-mode in tissue-mimetic agar phantoms. For these studies, pEM, 1D-pEM, and 2D-pEM formulations were prioritized based on their stability and initially evaluated for imaging contrast in the absence of the macrophage host (FIGS. 37-38). Results show the highest contrast is obtained from the 1D-pEM particles, achieving a 2.4-fold enhancement in signal relative to the control of media alone. Given that, among all nanoparticles tested, 1D-pEM particles show optimal stability (FIG. 23), cellular persistence (FIG. 32), and imaging contrast (FIG. 37), this formulation was prioritized for further investigation of macrophage imaging.

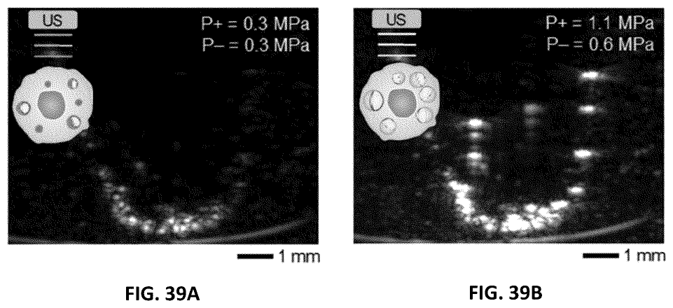

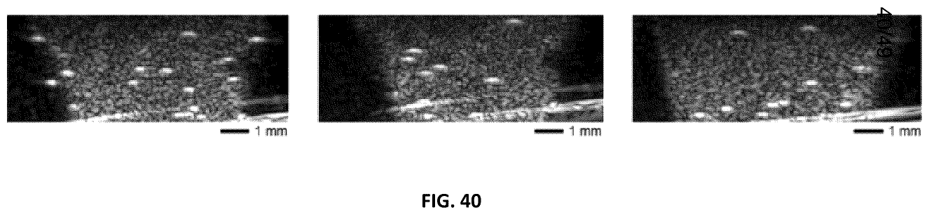

B-mode US imaging of RAW 264.7 cells loaded with 1D-pEM (hereafter referred to as 1D-pEM-RAW) was performed by pre-treating cells with the nanoparticles for 12 h, loading the contrast-enhanced cells into agar phantoms, and then submerging the sealed phantom into a 37° C. degassed water tank. The vaporization threshold was then measured by gradually increasing the peak positive and negative pressures (FIGS. 39A-39B) until a significant change of contrast was observed. As shown in FIG. 39A, B-mode imaging at low negative pressure of 0.3 MPa produced weak US signals due to the majority of nanoparticles remaining in the liquid phase. Increasing the peak negative pressure to 0.6 MPa induced a liquid-to-gas phase transition that generated echogenic microbubbles within the cells, yielding a significant enhancement in 1D-pEM-RAW cell contrast (FIG. 39B). Similar studies in blood indicated that, although background echogenicity is increased, 1D-pEM-RAW cells could still be clearly resolved from the noise (FIG. 40). In some cells, a sufficient number of intracellular microbubbles were created to cause the macrophages to become buoyant and float to the media surface. It is possible that, once outside of the imaging field, these cells may lose their buoyancy as the bubbles collapse or recondense back into liquid emulsions. Importantly, the mechanical index (0.14 at P−=0.6 MPa), the spatial peak temporal average intensity (8 mW cm-2 at P+=1.1 MPa), and the spatial peak pulse average intensity (10 W cm-2 at P+=1.1 MPa) required to generate high contrast B-mode images of 1D-pEM-RAW are all below the FDA diagnostic limit of 1.9, 720 mW cm-2, and 190 W cm-2, respectively, demonstrating the safety and translatability of this platform.

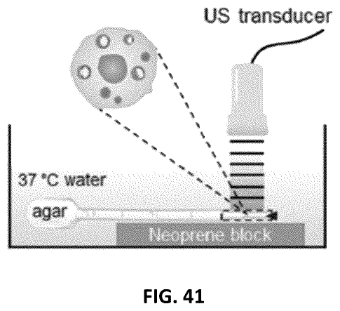

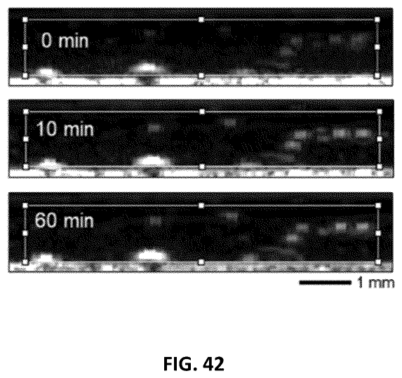

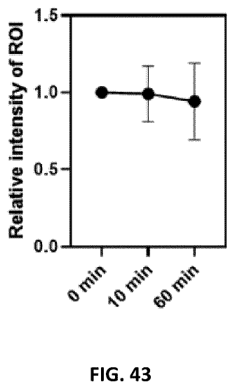

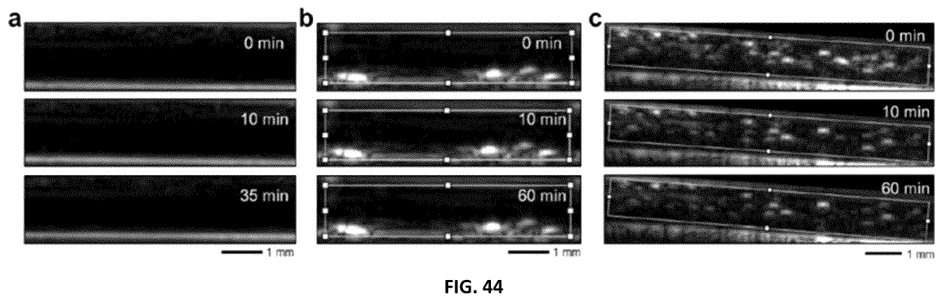

Next, to evaluate the persistence of imaging contrast during long-term US surveillance, the B-mode intensity of 1D-pEM-RAW cells was monitored over a 60 min continuous insonation period (FIGS. 41-44). Remarkably, a statistically significant attenuation in the signal over the entire 1 h imaging time was not observed. This is in stark contrast to conventional microbubbles that have imaging half-lives of ≈2-20 min. These findings suggest that the low negative acoustic pressure (0.4 MPa) used to induce vaporization of the emulsions permits stable bubble oscillation, as opposed to explosive cavitation, to provide persistent contrast without inducing macrophage damage. Together, this demonstrates the feasibility of prolonged and repeated US imaging of nanoparticle-loaded macrophages with high spatiotemporal resolution.

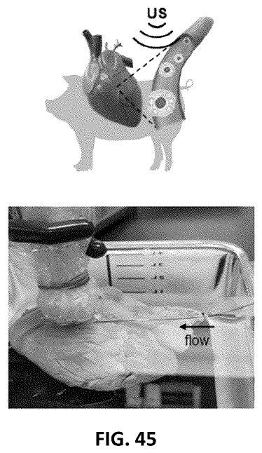

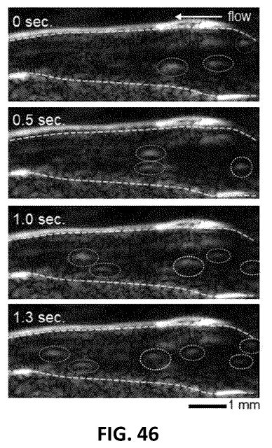

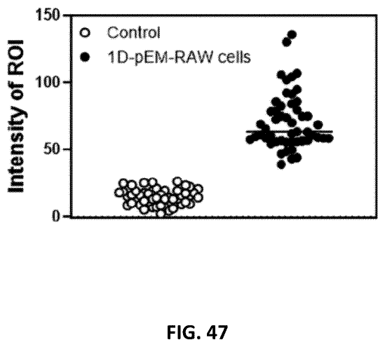

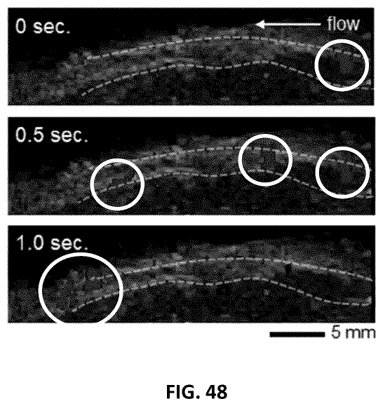

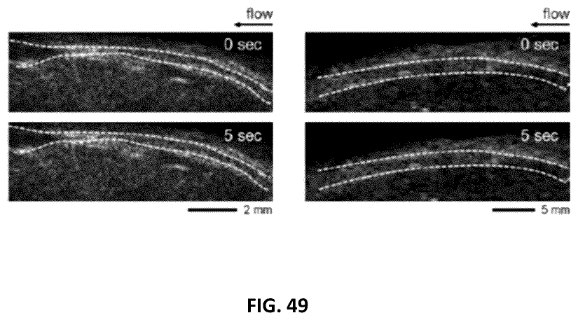

To further validate this platform in tissues the real-time imaging performance of 1D-pEM-RAW cells circulating within the vasculature of a porcine heart, or unloaded cells as control, was assessed (FIGS. 45-49). Here, the coronary artery was catheterized, and a degassed glycerol solution loaded with 1D-pEM-RAW cells was injected via a syringe pump at a flow rate of 1 mL min-1. Contrary to the control (FIG. 49), contrast enhanced 1D-pEM-RAW cells were clearly visualized under B-mode imaging as they perfused through the vessel, allowing single cell tracking and monitoring (see colored circles in FIG. 46). Quantification of the B-mode signal demonstrated a 4.7-fold increase in the contrast of 1D-pEM-RAW cells relative to the tissue background (FIG. 47). In separate experiments, color Doppler US (5 MHz) was employed to evaluate the potential of this modality to further improve the spatiotemporal resolution of 1D-pEM-RAW cell imaging (FIG. 48). Here, a frequency shift in the acoustic signal reflected from the surface of oscillating bubbles is interpreted by the US transducer to provide a colored feature that enables clearer differentiation of the contrast nuclei from the surrounding tissue. As shown in FIG. 48, Doppler signals were observed throughout the vessel during perfusion with 1D-pEM-RAW cells. These color features are ascribed to the circulating 1D-pEM-RAW cells since control vessels perfused with unloaded cells did not show similar signals (FIG. 49), and separate experiments demonstrated that free emulsions themselves provide strong Doppler contrast during stable oscillation in agar. These findings are further validated from prior studies that report microbubbles phagocytosed by leukocytes produce a higher mean frequency echo relative to free bubbles due to intracellular viscous dampening. The results substantiate the ability of peptide nanoparticles to enable contrast-enhanced in situ US imaging of macrophages in a real-time, continuous, and precise manner.

What is claimed is:

1. A composition comprising a plurality of cells,
wherein each cell comprises at least one peptide-based nanoparticle,
wherein the at least one peptide-based nanoparticle comprises a perfluorocarbon liquid core and a plurality of amphiphilic peptides surrounding the perfluorocarbon liquid core,
wherein each amphiphilic peptide is represented by Formula (III): 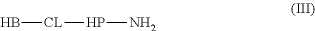
wherein HB is a fluorinated hydrophobic block consisting of three to five consecutively connected pentafluorinated hydrophobic amino acid residues;
wherein CL is an amino acid sequence consisting of two to 10 amino acid residues, at least two of which are cross-linking cysteine residues;
wherein HP is a hydrophilic amino acid sequence,
wherein said amphiphilic peptide consists of 8 to 30 total amino acid residues,
wherein the amphiphilic peptides are oriented such that groups HB of the amphiphilic peptides are interpolated into the perfluorocarbon liquid of the perfluorocarbon liquid core and groups HP extend away from the perfluorocarbon liquid core.

2. The composition of claim 1, wherein each cell of the plurality of cells is a macrophage.

3. The composition of claim 1, wherein the amphiphilic peptides of each peptide-based nanoparticle are crosslinked to each other through the cross-linking motif CL.

4. The composition of claim 3, wherein each peptide-based nanoparticle comprises a crosslinked unimolecular monolayer morphology or a 2D sheet morphology.

5. The composition of claim 1, wherein the amphiphilic peptides of each peptide-based nanoparticle are not crosslinked to each other.

6. The composition of claim 5, wherein each peptide-based nanoparticle comprises a non-crosslinked unimolecular monolayer morphology or a ID fibrils morphology.

7. The composition of claim 1, wherein the composition further comprises a pharmaceutically acceptable excipient selected from the group consisting of a vehicle, an adjuvant, a carrier, and a diluent.

8. The composition of claim 1, wherein HB consists of three, four or five consecutively connected pentafluoro-phenylalanine residues, and is located at the N-terminal end of the peptide sequence.

9. The composition of claim 1, wherein HP comprises lysine, glycine, arginine, aspartic acid, or any combination thereof.

10. The composition of claim 1, wherein HP comprises the sequence KGRGD (SEQ ID NO: 35), where K is lysine, G is glycine, R is arginine, and D is aspartic acid.

11. The composition of claim 1, wherein CL comprises GGGCCGG (SEQ ID NO: 46), where G is glycine and C is cysteine.

12. The composition of claim 1, wherein said hydrophilic amino acid sequence of HP comprises a targeting motif.

13. The composition of claim 1, wherein said hydrophilic amino acid sequence comprises a conserved targeting motif selected from the group consisting of: HGK, RGD, KAR, RSR, KAA, RGRR (SEQ ID NO:1), RGRRS (SEQ ID NO:2), YQLDV (SEQ ID NO:3), EYQ, RPM, PSP, VGVA (SEQ ID NO:4), NGR, CRKRLDRNC (SEQ ID NO:43), EFEEFEIDEEEK (SEQ ID NO:44), and DFEEIPEEYLQ (SEQ ID NO:45).

14. The composition of claim 1, wherein said hydrophilic amino acid sequence comprises a hydrophilic amino acid sequence selected from the group consisting of: KGRGD (SEQ ID NO:35), RGDS (SEQ ID NO:36), GRGD (SEQ ID NO:37), GRGDS (SEQ ID NO:38), GRGDSP (SEQ ID NO:39), GRGDSPK (SEQ ID NO:40), GRGDNP (SEQ ID NO:41), and GRGDTP (SEQ ID NO:42).

15. The composition of claim 1, wherein said amphiphilic peptide comprises an amphiphilic peptide represented by Formula (IV) or Formula (V): F_{F}F_{F}F_{F}GGGCCGGKGRGD (IV) (SEQ ID NO:47), F_{F}F_{F}F_{F}F_{F}GGGCCGGKGRGD-NH2 (V) (SEQ ID NO:49), wherein F_{F} is pentafluoro-phenylalanine, G is glycine, C is cysteine, K is lysine, G is glycine, R is arginine, and D is aspartic acid.

16. The composition of claim 1, wherein said amino acid sequence of CL consists of two to 10 amino acid residues and said hydrophilic amino acid sequence of HP consists of 3 to 15 hydrophilic amino acids, and wherein said amphiphilic peptide consists of 10 to 30 total amino acid residues.

17. The composition of claim 1, wherein the amphiphilic peptide has a molecular weight in a range of about 2000-5000 daltons, wherein the amphiphilic peptide comprises at least eight amino acid residues, and a total number of no more than 30 amino acid residues, wherein at least two of the amino acid residues are connected consecutively by peptide bonds without any intervening amino acid residues.

18. A composition comprising a plurality of macrophages,
wherein each macrophage comprises at least one peptide-based nanoparticle,
wherein the at least one peptide-based nanoparticle comprises a perfluorocarbon liquid core and a plurality of amphiphilic peptides surrounding the perfluorocarbon liquid core,
wherein each amphiphilic peptide comprises F_{F}F_{F}F_{F}F_{F}GGGCCGGKGRGD-NH2 (SEQ ID NO: 49), wherein F_{F} is pentafluoro-phenylalanine, G is glycine, C is cysteine, K is lysine, G is glycine, R is arginine, and D is aspartic acid, and
wherein amphiphilic peptides are oriented such that the pentafluoro-phenylalanine region is interpolated into the perfluorocarbon liquid core and the KGRGD (SEQ ID NO:35) region extends away from the perfluorocarbon liquid.

19. The composition of claim 18, wherein the amphiphilic peptides of each peptide-based nanoparticle are crosslinked to each other through a cross-linking motif GGGCCGG (SEQ ID NO: 46), and wherein each peptide-based nanoparticle comprises a crosslinked unimolecular monolayer morphology or a 2D sheet morphology.

20. The composition of claim 18, wherein the amphiphilic peptides of each peptide-based nanoparticle are not crosslinked to each other, and wherein each peptide-based nanoparticle comprises a non-crosslinked unimolecular monolayer morphology or a 1D fibrils morphology.

21. A method of preparing the cellular composition comprising a plurality of cells of claim 1, the method comprising:
contacting a perfluorocarbon liquid with a plurality of amphiphilic peptides to form a plurality of peptide-based nanoparticles, wherein each peptide-based nanoparticle comprises a perfluorocarbon liquid core and a plurality of amphiphilic peptides surrounding the perfluorocarbon liquid core, and
contacting the plurality of peptide-based nanoparticles with the plurality of cells, wherein each cell of the plurality of cells internalizes at least one peptide-based nanoparticle.

22. The method of claim 21, wherein water is added to plurality of peptide-based nanoparticles after their formation.

23. The method of claim 22, wherein the amphiphilic peptides of each peptide-based nanoparticle are crosslinked to each other, forming a peptide-based nanoparticle comprising a crosslinked unimolecular monolayer morphology.

24. The method of claim 22, wherein the amphiphilic peptides of each peptide-based nanoparticle are not crosslinked to each other, forming a peptide-based nanoparticle comprising a non-crosslinked unimolecular monolayer morphology.

25. The method of claim 21, wherein water is added to the plurality of amphiphilic peptides prior to their contact with the perfluorocarbon liquid.

26. The method of claim 25, wherein the amphiphilic peptides of each peptide-based nanoparticle are crosslinked to each other, forming a peptide-based nanoparticle comprising a 2D sheet morphology.

27. The method of claim 25, wherein the amphiphilic peptides of each peptide-based nanoparticle are not crosslinked to each other, forming a peptide-based nanoparticle comprising a 1D fibrils morphology.

28. A method of cellular tracking, comprising:
administering a composition of claim 1 to a tissue,
administering ultrasonic waves to the tissue, and
detecting the location of the cells in the tissue by locating acoustic properties of the peptide-based nanoparticles in the plurality of cells.

29. The method of claim 28, wherein the ultrasonic waves are administered to the tissue by a B-mode ultrasonic imaging device or a Doppler ultrasonic imaging device.

30. The method of claim 28, wherein the ultrasonic waves induce a liquid-to-gas phase transition in the peptide-based nanoparticles that generates echogenic microbubbles inside the plurality of cells.
